SEQUENCE LISTING (9 sequences)

SEQ ID NO: 1 - DNA, 23 bp, Artificial
  GFP-targeting sgRNA
ccagggcacgggcagcttgccgg

SEQ ID NO: 2 - DNA, 23 bp, Artificial
  GFP-targeting sgRNA
ggcgagggcgatgccacctacgg

SEQ ID NO: 3 - DNA, 23 bp, Artificial
  FGFR3-targeting sgRNA
acgcggccaagggtaacctgcgg

SEQ ID NO: 4 - DNA, 23 bp, Artificial
  FGFR3-targeting sgRNA
gaaggagtagtccaggcccgggg

SEQ ID NO: 5 - DNA, 23 bp, Artificial
  FGFR3-targeting sgRNA
aggtgtcgaaggagtagtccagg

SEQ ID NO: 6 - DNA, 23 bp, Artificial
  TACC3-targeting sgRNA
cggagtctccgctttgtgctggg

SEQ ID NO: 7 - DNA, 23 bp, Artificial
  TACC3-targeting sgRNA
agaccctagaagacccttgcagg

SEQ ID NO: 8 - DNA, 23 bp, Artificial
  TACC3-targeting sgRNA
gccgaggaggaatgccggcacgg

SEQ ID NO: 9 - DNA, 23 bp, Artificial
  F-T-targeting sgRNA
ttcgagcagtactccccgggtgg

(19) United States
(12) Patent Application Publication
(10) Pub. No.: US 2022/0087947 A1
(43) Pub. Date: Mar. 24, 2022

(54) NANOPARTICLE FOR DELIVERING RIBONUCLEOPROTEIN AND USE THEREOF

(71) Applicant: KOREA INSTITUTE OF SCIENCE AND TECHNOLOGY, Seoul (KR)

(72) Inventors: Hyojin LEE, Seoul (KR); Seongchan KIM, Seoul (KR); Youngdo JEONG, Seoul (KR)

(73) Assignee: KOREA INSTITUTE OF SCIENCE AND TECHNOLOGY, Seoul (KR)

(21) Appl. No.: 17/384,913

(22) Filed: Jul. 26, 2021

(30) Foreign Application Priority Data
Sep. 17, 2020 (KR) 10-2020-0120002

(51) Int. Cl. A61K 9/51; C12N 9/22; C12N 15/11

(52) U.S. Cl. CPC A61K 9/5115; C12N 15/111; C12N 9/22

(57) ABSTRACT

Provided are a surface-aminated mesoporous silica nanoparticle, a method of preparing the same, use of the nanoparticle for delivering a ribonucleoprotein, and use of the nanoparticle for the prevention or treatment of cancer. The surface-aminated mesoporous silica nanoparticle may be loaded with large RNPs such as Cas9-RNP, and thus may be applied to treatment of various diseases treatable by gene editing.

### CROSS-REFERENCE TO RELATED APPLICATION

This application is based on and claims priority under 35 U.S.C. § 119 to Korean Patent Application No. 10-2020-0120002, filed on Sep. 17, 2020, in the Korean Intellectual Property Office, the disclosure of which is incorporated by reference herein in its entirety.

### BACKGROUND

### 1. Field

The present disclosure relates to a surface-aminated mesoporous silica nanoparticle, a method of preparing the nanoparticle, use of the nanoparticle for delivering a ribonucleoprotein, and use of the nanoparticle for the prevention or treatment of cancer.

### 2. Description of the Related Art

Gene fusion in neoplasia usually occurs through chromosomal rearrangement of two or more independent genes, inducing tissue abnormality and tumor development. Therefore, to understand pathological mechanisms of cancer and to treat diseases, many studies focus on determining how fusion genes control the function, growth, and differentiation of cancer cells.

Among the fusion genes, the genes consisting of fibroblast growth factor receptor 3 (FGFR3) and transforming acidic coiled-coil containing protein 3 (TACC3) have been identified in various cancer patients such as those with glioblastoma, lung adenocarcinoma, squamous cell lung carcinoma, head and neck squamous cell carcinoma, bladder cancer, cervical cancer, breast invasive ductal carcinoma, urothelial carcinoma, malignant glioma, and glioma, and have been significantly associated with tumorigenesis. The expression of the fusion genes and proteins shows higher tissue-specificity than other proteins due to their tumor-specific expression, thereby providing immense diagnostic and therapeutic advantages.

The FGFR3-TACC3 (F-T) fusion gene preserves the gene expression patterns of both FGFR3 and TACC3, and is highly expressed in tumors as compared to normal tissue. It also activates oncogenic pathways, including mitogen-activated protein kinase (MAPK)/extracellular-regulated kinase (ERK) and phosphatidylinositol 3-kinase (PI3K)/Akt signaling, which are also highly correlated with the original role of FGFR3 and TACC3, respectively. Although the function of the F-T fusion gene is highly similar to that of the FGFR3 and TACC3 genes, patients with the F-T fusion gene show restricted sensitivity to traditional FGFR inhibitor drugs. Therefore, new alternative approaches such as multigene (FGFR3 and TACC3) editing and the development of new inhibitors are required for therapy. However, multigene engineering strategies are limited because of few reliable practical platforms and, to date, gene therapies associated with such strategies are lacking as an alternative to the classical therapeutic approaches such as use of viruses, plasmids, and siRNA.

Here, the present inventors applied and optimized CRISPR/Cas9 technology for efficient multigene engineering. There are a few methods for gene editing using the CRISPR/Cas9 technology. Among these, the Cas9/single-guide RNA (sgRNA) ribonucleoprotein (Cas9-RNP) has some advantages for application in vivo due to its greater specificity and lower toxicity than Cas9 plasm id DNA/sgRNA and Cas9 mRNA/sgRNA complexes. Furthermore, Cas9-RNP is able to circumvent the cellular transcriptional or translational stage, which allows for rapid and efficient gene editing. Although the merits of Cas9-RNP are obvious, its application still faces some limitations, such as low stability due to enzymatic degradation, low permeability owing to strong negative charges, large molecular size (>160 kDa), and endosomal entrapment of Cas9-RNP inside the cells. These factors limit the accumulation of Cas9-RNP in specific organs after systemic administration, and eventually, reduce the efficacy of targeted gene editing in vivo. Accordingly, there is a need for a facile and convenient strategy for efficient intracellular Cas9-RNP delivery.

### PRIOR ART DOCUMENTS

### Non-Patent Documents

(Non-Patent Document 1) Kunwoo Lee et al., “Nanoparticle delivery of Cas9 ribonucleoprotein and donor DNA in vivo induces homology-directed DNA repair”, Nat Biomed Eng. 2017; 1: 889-901.

### SUMMARY

There is provided a method of preparing a surface-aminated mesoporous silica nanoparticle.

There is provided a method of loading a ribonucleoprotein (RNP) onto the surface-aminated mesoporous silica nanoparticle.

There is provided a surface-aminated mesoporous silica nanoparticle.

There is provided a composition including the surface-aminated mesoporous silica nanoparticle.

There is provided a pharmaceutical composition for preventing or treating cancer, the pharmaceutical composition including the surface-aminated mesoporous silica nanoparticle.

There is provided a method of preventing or treating a disease, the method including administering an effective amount of the pharmaceutical composition to a subject in need thereof.

Additional aspects will be set forth in part in the description which follows and, in part, will be apparent from the description, or may be learned by practice of the presented embodiments of the disclosure.

An aspect provides a method of preparing a surface-aminated mesoporous silica nanoparticle.

The preparation method includes (1) dissolving a cationic surfactant in a basic solution;

(2) adding a silica precursor solution to the solution prepared in (1) and stirring the mixture to prepare porous silica nanoparticles;

(3) adding a benzenoid compound solution to the porous silica nanoparticles and allowing them to react to prepare mesoporous silica nanoparticles; and

(4) adding an amine compound solution to the mesoporous silica nanoparticles to prepare a mixture and refluxing the mixture to prepare the surface-aminated mesoporous silica nanoparticles.

(1) is a step of dissolving a cationic surfactant in a basic solution.

In (1), the cationic surfactant may be a structure-directing agent (SDA).

In (1), the cationic surfactant is not limited to its kind, as long as it is a common cationic surfactant which may be used as a structure-directing agent of nanoparticles. In one specific embodiment, in (1), the cationic surfactant may be cetyl trimethyl ammonium bromide (CTAB) or cetyl trimethyl ammonium chloride (CTAC). In (1), the cationic surfactant may be, for example, CTAB.

In (1), the basic solution may have pH of 9 to 13. The basic solution may be a solution including NaOH, KOH, NH₄OH, or Tris, but is not limited thereto.

(2) is a step of adding a silica precursor solution to the solution prepared in (1) and stirring the mixture to prepare porous silica nanoparticles. The porous silica nanoparticles prepared in (2) may be monodisperse porous silica nanoparticles. The porous silica nanoparticles may have a mean pore size of 1 nm to 5 nm, for example, a mean pore size of about 3 nm. The porous silica nanoparticles may have a diameter of 40 nm to 1000 nm, 40 nm to 800 nm, 40 nm to 500 nm, 40 nm to 400 nm, 40 nm to 300 nm, 100 nm to 1000 nm, 100 nm to 800 nm, 100 nm to 500 nm, 100 nm to 400 nm, 100 nm to 300 nm, 150 nm to 1000 nm, 150 nm to 800 nm, 150 nm to 500 nm, 150 nm to 400 nm, or 150 nm to 300 nm, for example, a diameter of about 200 nm.

In (2), the silica precursor may be tetramethyl orthosilicate, (TMOS), tetraethyl orthosilicate (TEOS), (3-glycidoxypropyl)methyldiethoxysilane (GPTMS), 3-mercaptopropyl trimethoxysilane (MPS), γ-glycidyloxypropyl trimethoxysilane (GOTMS), or aminophenyl trimethoxysilane (APTMOS). The silica precursor may be TMOS or TEOS.

The silica precursor may be in a liquid form at room temperature. The silica precursor solution may be obtained by dissolving the silica precursor in water, alcohol, or a mixture thereof. The alcohol may be C1 to C10 alcohol. The alcohol may be, for example, ethanol or methanol.

In (2), the silica precursor solution may be added at a flow rate of 0.1 mL/h to 10.0 mL/h, 0.1 mL/h to 5.0 mL/h, 0.1 mL/h to 1.0 mL/h, 0.3 mL/h to 10.0 mL/h, 0.3 mL/h to 5 mL/h, 0.3 mL/h to 1.0 mL/h, or 0.3 mL/h to 0.7 mL/h. For example, a TMOS solution may be added at a flow rate of 0.5 mL/h. The addition of the TMOS solution may be performed using a tube-connected syringe pump, but any device capable of adding the solution at a predetermined flow rate may be used without limitation in the kind thereof. Excessively slow or fast flow rates may affect nanoparticle size and pore formation.

In (2), the stirring may be performed for 1 hour to 24 hours, 1 hour to 20 hours, 1 hour to 16 hours, 1 hour to 12 hours, 1 hour to 8 hours, 2 hours to 24 hours, 2 hours to 20 hours, 2 hours to 16 hours, 2 hours to 12 hours, 2 hours to 8 hours, 4 hours to 24 hours, 4 hours to 20 hours, 4 hours to 16 hours, 4 hours to 12 hours, or 4 hours to 8 hours, but is not limited thereto.

(2) may further include leaving the mixture for 1 hour to 24 hours without stirring, after performing the stirring.

The method may further include centrifuging the porous silica nanoparticles, after (2).

The method may further include washing the porous silica nanoparticles, after (2). Specifically, the washing may be washing the centrifuged porous silica nanoparticles. The washing may be performed by an appropriate method selected by those skilled in the art. For example, alcohol, water, or a mixture thereof may be used. In one specific embodiment, the washing may be performed using ethanol and water, but is not limited thereto.

(3) is adding the porous silica nanoparticles to a benzenoid compound solution and allowing them to react to prepare mesoporous silica nanoparticles. The mesoporous silica nanoparticles prepared in (3) may have a mean pore size of 15 nm to 50 nm, for example, a mean pore size of about 20 nm. Unlike general mesoporous nanoparticles having a mean pore size of 2 nm to 50 nm, the mesoporous silica nanoparticles prepared according to one specific embodiment may have a mean pore size of 15 nm or more, or 20 nm or more, which is a very large pore size. The porous silica nanoparticles may have a diameter of 40 to 1000 nm, 40 to 800 nm, 40 to 500 nm, 40 to 400 nm, 40 to 300 nm, 100 to 1000 nm, 100 to 800 nm, 100 to 500 nm, 100 to 400 nm, 100 to 300 nm, 150 to 1000 nm, 150 to 800 nm, 150 to 500 nm, 150 to 400 nm, or 150 to 300 nm, for example, a diameter of about 200 nm.

In (3), the benzenoid compound may be any one or more selected from the group consisting of benzene, trimethylbenzene (TMB), 1,3,5-triethylbenzene (TEB), 1,3,5-tri-tert-butylbenzene (TtBB), and 1,3,5-Triisopropylbenzene (TiPB). For example, the benzenoid compound may be TMB.

In (3), the benzenoid compound solution may be a mixture of a benzenoid compound, alcohol, and distilled water. A volume ratio of the benzenoid compound, alcohol, and distilled water may be 1-2:1-2:1-2, for example, 1:1:1, but is not limited thereto. The alcohol may be, for example, ethanol, but is not limited thereto.

In (3), the reaction may be performed under a high temperature condition. In (3), the reaction may be performed at 120° C. to 200° C., 120° C. to 180° C., 120° C. to 160° C., 140° C. to 200° C., 140° C. to 180° C., or 140° C. to 160° C. The reaction may be performed, for example, at 160° C. When the reaction temperature is excessively low or high, pore expansion of nanoparticles may hardly occur, or uniform pore expansion may be difficult. Further, the structure of the particles may collapse.

In (3), the reaction may be performed for 24 hours to 72 hours, 24 hours to 60 hours, 24 hours to 48 hours, 36 hours to 72 hours, 36 hours to 60 hours, 36 hours to 48 hours, 48 hours to 72 hours, or 48 hours to 60 hours. The reaction may be performed, for example, for 48 hours. When the reaction time is excessively short or long, pore expansion of nanoparticles may hardly occur, or uniform pore expansion may be difficult. Further, the structure of the particles may collapse.

In (3), the reaction may be performed in any reaction vessel. The reaction may be performed, for example, in an autoclave or a furnace, but is not limited thereto.

The method may further include washing the mesoporous silica nanoparticles, after (3). The washing may be performed by an appropriate method selected by those skilled in the art. For example, alcohol, water, or a mixture thereof may be used. In one specific embodiment, the washing may be performed using ethanol and water, but is not limited thereto.

The method may further include adding an acidic compound to the mesoporous silica nanoparticles and refluxing the mixture to remove the cationic surfactant, after (3).

The acidic solution may have pH of 1 to 5. The acidic solution may include HCl, but is not limited thereto.

The refluxing may be performed at 60° C. to 180° C., 60° C. to 150° C., 90° C. to 180° C., 90° C. to 150° C., 100° C. to 180° C., 100° C. to 150° C., 100° C. to 140° C., 100° C. to 130° C., 100° C. to 120° C., 110° C. to 180° C., 110° C. to 150° C., 110° C. to 140° C., or 110° C. to 130° C.

(4) is adding an amine compound solution to the mesoporous silica nanoparticles to prepare a mixture and refluxing the mixture to prepare the surface-aminated mesoporous silica nanoparticles. The mesoporous silica nanoparticles may be functionalized with positively charged groups by (4). The surface charge of the nanoparticles may be controlled by (4). Therefore, the surface-aminated mesoporous silica nanoparticles may be loaded with a negatively charged substance through an electrostatic interaction.

In (4), the amine compound is not limited to its kind, as long as it may functionalize the silica nanoparticles with positively charged groups. The amine compound may be a silane compound having amine (NH₂). The amine compound may be, for example, any one or more selected from the group consisting of N(beta-aminoethyl)gamma-aminopropylmethyldimethoxysilane (AEAPMDMS), N-[3-(trimethoxysilyl)propyl]ethylenediamine, N1-(3-trimethoxysilylpropyl)diethylenetriamine, (3-aminopropyl)trimethoxysilane, N-[3-(trimethoxysilyl)propyl]aniline, trimethoxy[3-(methylamino)propyl]silane, 3-(2-aminoethylamino)propyldimethoxymethylsilane, trimethoxysilylpropyl modified polyethylenimine (TPPI), p-aminophenyltrimethoxysilane, and 1-amino-2-(dimethylethoxysilyl)propane.

The refluxing may be performed at 60° C. to 180° C., 60° C. to 150° C., 90° C. to 180° C., 90° C. to 150° C., 100° C. to 180° C., 100° C. to 150° C., 100° C. to 140° C., 100° C. to 130° C., 100° C. to 120° C., 110° C. to 180° C., 110° C. to 150° C., 110° C. to 140° C., or 110° C. to 130° C.

The method may further include washing the surface-aminated mesoporous silica nanoparticles, after (4). The washing may be performed by an appropriate method selected by those skilled in the art. For example, alcohol, water, or a mixture thereof may be used. In one specific embodiment, the washing may be performed using ethanol and water, but is not limited thereto.

Another aspect provides a method of loading a negatively charged substance onto the surface-aminated mesoporous silica nanoparticles, the method including mixing the surface-aminated mesoporous silica nanoparticles prepared by the preparation method according to one aspect with the negatively charged substance. In other words, the method may be a method of preparing a complex of the surface-aminated mesoporous silica nanoparticle and the negatively charged substance.

Still another aspect provides a method of loading a ribonucleoprotein (RNP) onto the surface-aminated mesoporous silica nanoparticles, the method including mixing the surface-aminated mesoporous silica nanoparticles prepared by the preparation method according to one aspect with the RNP. In other words, the method may be a method of preparing a complex of the surface-aminated mesoporous silica nanoparticle and the RNP.

The mixing may be performed at a mild temperature. In one specific embodiment, the mixing may be performed at room temperature. The room temperature has the meaning of room temperature commonly used in the art, and it may mean, for example, 1° C. to 35° C.

The mixing may be performed at a mild temperature. In one specific embodiment, the mixing may be bringing the nanoparticles into contact with the negatively charged substance (e.g., RNP) in any solution for 10 minutes to 3 hours, 10 minutes to 2 hours, 10 minutes to 1 hour, 20 minutes to 3 hours, 20 minutes to 2 hours, or 20 minutes to 1 hour.

Therefore, the mixing may be performed at room temperature for 10 minutes to 3 hours.

The mixing may allow the surface-aminated mesoporous silica nanoparticles to be loaded with the negatively charged substance (e.g., RNP) by an electrostatic interaction of the nanoparticle and the negatively charged substance.

The method of loading the negatively charged substance (e.g., RNP) onto the surface-aminated mesoporous silica nanoparticles according to one aspect may be performed under mild conditions, and thus when the substance loaded inside thereof includes an enzyme such as Cas9, the enzymatic activity may be maintained.

Still another aspect provides the surface-aminated mesoporous silica nanoparticles.

The nanoparticles may have a mean pore size of 15 nm or more or 20 nm or more, and may have a mean pore size of 15 nm to 50 nm, 15 nm to 40 nm, 15 nm to 30 nm, 15 nm to 25 nm, 20 nm to 50 nm, 20 nm to 40 nm, 20 nm to 30 nm, 20 nm to 25 nm, 20 nm to 23 nm, 20 nm to 22 nm, or 20 nm to 21 nm. Therefore, the nanoparticles according to one aspect have a larger pore size than a general mesoporous particle having a mean pore size of 2 nm to 50 nm. For this reason, the nanoparticles may be loaded with a large substance.

The surface-aminated mesoporous silica nanoparticles may be loaded with a ribonucleoprotein (RNP). Therefore, the surface-aminated mesoporous silica nanoparticles may be for RNP delivery.

The nanoparticles may have a diameter of 150 nm or more or 200 nm or more, and for example, a diameter of 150 nm to 1000 nm, 150 nm to 500 nm, 150 nm to 400 nm, 150 nm to 300 nm, 150 nm to 250 nm, 180 nm to 500 nm, 180 nm to 400 nm, 180 nm to 300 nm, 180 nm to 250 nm, 180 nm to 220 nm, 200 nm to 500 nm, 200 nm to 400 nm, 200 nm to 300 nm, 200 nm to 250 nm, or 200 nm to 220 nm. Therefore, the nanoparticles according to one aspect have a larger diameter than general nanoparticles having a diameter of 100 nm or less.

The nanoparticles may have a controlled surface charge. The nanoparticles may have a zeta potential of +20.0 mV to +30.0 mV, and for example, a zeta potential of +20.0 mV to +28.0 mV, +20.0 mV to +26.0 mV, +22.0 mV to +30.0 mV, +22.0 mV to +28.0 mV, +22.0 mV to +26.0 mV, +24.0 mV to +30.0 mV, +24.0 mV to +28.0 mV, +24.0 mV to +26.0 mV, or +24.0 mV to +25.0 mV.

The nanoparticles may be loaded with a larger negatively charged substance due to the above-described pore size, diameter, and zeta potential characteristics. For example, even though Cas9-RNP has a large size of 160 kDa, and is a negatively charged substance, it may be loaded onto the nanoparticles according to one aspect with high capacity.

The nanoparticles may be for release control. The nanoparticles may have an RNP loading capacity of about 10% (w/w) to about 20% (w/w), for example, about 10% (w/w) to about 15% (w/w) at a neutral pH, specifically, at pH 7.0 to pH 8.0, for example, at pH 7.4. The nanoparticles according to one embodiment were confirmed to have a Cas9-RNP loading capacity of about 12.5% (w/w) at pH 7.4, indicating very excellent loading capacity.

The nanoparticles may have a higher drug release rate under an acidic pH condition of pH 6.5 or less, specifically, at pH 3.0 to pH 6.5, for example, under an acidic pH condition of pH 5.0 to pH 6.0 than a drug release rate of the nanoparticles under a neutral pH condition of pH 7.0 to pH 8.0. In one specific embodiment, the nanoparticles may exhibit a drug release rate of 5% or less under a neutral pH condition of pH 7.0 to pH 8.0 in 12 hours, and a drug release rate of 30% or more under an acidic pH condition of pH 6.5 or less in 12 hours. In another specific embodiment, the nanoparticles may exhibit a drug release rate of 5% or less at about pH 7.4 in 12 hours, a drug release rate of 30% or more at about pH 6.0 in 12 hours, and a drug release rate of 70% or more at about pH 5.0 in 12 hours. Therefore, the nanoparticles may release RNP to cytoplasm in vivo. This indicates that the release rate of RNP increases under the acidic environment which is close to the endosomal conditions. Further, the sustained release property of the nanoparticles according to one aspect indicates that Cas9 protein activity may be maintained with high stability for a long time under in vivo environment.

The surface-aminated mesoporous nanoparticles may be for delivering a gene-editing material. Genome editing or gene editing may be a method of modifying a target nucleic acid in the genome of a cell or living organism. The gene editing may be performed using a clustered regularly interspaced repeat/CRISPR-associated (CRISPR/Cas) system, a base editor, zing finger nuclease (ZFN), transcriptional activator-like effector nuclease (TALEN), or RNA-guided engineered nuclease (RGEN). For example, the surface-aminated mesoporous nanoparticles may be for genetic scissors delivery. The genetic scissors refers to a restriction enzyme that recognizes a specific nucleotide sequence in the genome and cuts DNA at the corresponding site.

The gene-editing material may be a nucleic acid for gene editing, a protein for gene editing, or a combination thereof. The gene-editing material may include a Cas protein or a variant thereof, a nucleic acid encoding the same or a nucleic acid complementary thereto, or a combination thereof. The gene-editing material may also include a nucleic acid encoding a guide RNA or a nucleic acid complementary thereto, or a combination thereof.

The RNP may be a Cas protein-RNP. The Cas protein may be Cas9, nickase Cas9 (nCas9), deactivated Cas9 (dCas9), nCas9 fused to cytidine deaminase (i.e. nCas9+cytidine deaminase), nCas9 fused to adenine deaminase (i.e. nCas9+adenine deaminase), dCas9 fused to cytidine deaminase (i.e. dCas9+cytidine deaminase), dCas9 fused to adenine deaminase (i.e. dCas9+adenine deaminase), Cas12, Cas13, Cpf1, C2c2, or a combination of two or more thereof, but is not limited thereto.

The Cas protein-RNP may be, for example, a CRISPR/Cas9 single-guide RNA (sgRNA) ribonucleoprotein, i.e., Cas9-RNP. The Cas9-RNP may include a guide RNA targeting a target gene in need of gene editing. The guide RNA sequence is not limited to a specific sequence.

For example, the Cas protein-RN P may be nCas9+deaminase sgRNA or dCas9-deaminase sgRNA ribonucleoprotein, i.e., Base editor-RNP. The Base editor-RNP may include a guide RNA targeting a target gene in need of gene editing. The guide RNA sequence is not limited to a specific sequence.

The nanoparticles may be loaded with two or more types of Cas protein-RNP. In one specific embodiment, the Cas-RNP may be one type, or two or more types. In another specific embodiment, the Cas protein-RNP may be two types. In still another specific embodiment, the Cas9-RNP may be two types. In still another specific embodiment, the Cas9-RNP may be two types, wherein any one type thereof may include sgRNA targeting fibroblast growth factor receptor 3 (FGFR3), and the other type thereof may include sgRNA targeting transforming acidic coiled-coil containing protein 3 (TACC3).

A sequence of the sgRNA targeting FGFR3 and a sequence of the sgRNA targeting TACC3 are disclosed, and may be prepared according to a common method by those skilled in the art. The sgRNA targeting FGFR3 may include or consist of, for example, any one nucleotide sequence of SEQ ID NOS: 3 to 5. Further, the sgRNA targeting TACC3 may include or consist of, for example, any one nucleotide sequence of SEQ ID NOS: 6 to 8.

The nanoparticles may be the surface-aminated mesoporous silica nanoparticles prepared by the preparation method according to one aspect, but is not limited thereto.

Still another aspect provides a complex of the surface-aminated mesoporous silica nanoparticle according to one aspect and RNP.

The complex may be obtained by loading the RNP onto the surface-aminated mesoporous silica nanoparticles. Specifically, the surface-aminated mesoporous silica nanoparticle and RNP are bound to each other by an electrostatic interaction.

The RNP may be Cas protein-RNP. The RNP may be Cas9-RNP or Base editor-RNP.

The Cas protein-RNP may be one type or two or more types. The Cas9-RNP may be one type or two or more types. When the Cas9-RNP is two or more types, the types of the guide RNA included in the Cas9-RNP may differ. For example, when the Cas9-RNP is two types, any one thereof may include a guide RNA targeting any specific gene, and the other thereof may include a guide RNA targeting a gene different therefrom.

Still another aspect provides a composition including the surface-aminated mesoporous silica nanoparticles according to one aspect.

The composition may be formulated with a carrier for administration to a subject by injection, implantation, or tissue engineering matrix. The carrier may be a pharmaceutically acceptable carrier. The pharmaceutically acceptable carrier may be, for example, saline, sterile water, Ringer's solution, buffered saline, a dextrose solution, a maltodextrin solution, glycerol, ethanol, human serum albumin (HSA), and a mixture of one or more thereof, and as needed, other common additives, such as antioxidants, buffers, bacteriostatic agents, etc. may be added.

The composition may be for RNP delivery. The RNP may be a Cas protein-RNP. The RNP may be Cas9-RNP or Base editor-RNP. Therefore, the composition may be for delivering gene-editing materials.

Still another aspect provides a pharmaceutical composition for preventing or treating cancer, the pharmaceutical composition including the surface-aminated mesoporous silica nanoparticles according to one aspect.

The term “preventing” includes inhibiting occurrence of a disease.

The term “treating” includes inhibition, alleviation, or elimination of development of a disease.

The nanoparticles may be loaded with RNP. Therefore, the pharmaceutical composition may include the complex of the surface-aminated mesoporous silica nanoparticle and RNP. Specifically, the nanoparticles may be loaded with a Cas protein-RNP. The nanoparticles may be loaded with a Cas9-RNP, a Base editor-RNP, or a combination thereof. The nanoparticles may be loaded with one type or two or more types of Cas9-RNP. The nanoparticles may be loaded with two types of Cas9-RNP.

In a specific embodiment, the Cas9-RNP loaded onto the nanoparticle is two types, wherein any one thereof may include sgRNA targeting FGFR3, and the other thereof may include sgRNA targeting TACC3.

The sgRNA targeting FGFR3 may include any one nucleotide sequence of SEQ ID NOS: 3 to 5. The sgRNA targeting TACC3 may include any one nucleotide sequence of SEQ ID NOS: 6 to 8.

The pharmaceutical composition may include the complex of the surface-aminated mesoporous silica nanoparticle and RNP as an active ingredient.

The term “including as an active ingredient” means that the active ingredient is added such that the above-mentioned effects may be obtained. In addition, this may include formulation in various forms by adding various ingredients as sub-components for drug delivery and stabilization, etc.

The pharmaceutical composition may have use of preventing or treating cancer associated with a gene targeted by Cas9-RNP. The cancer associated with the gene targeted by Cas9-RNP may be, for example, a cancer associated with FGFR3-TACC3 fusion gene. The cancer associated with the FGFR3-TACC3 fusion gene may be selected from the group consisting of glioblastoma, lung adenocarcinoma, squamous cell lung carcinoma, head and neck squamous cell carcinoma, bladder cancer, cervical cancer, breast invasive ductal carcinoma, urothelial carcinoma, malignant glioma, and glioma, but is not limited thereto (Cancer Discovery. 2017; 7(8):818-831.).

The pharmaceutical composition may further include a pharmaceutically acceptable diluent or carrier. The diluent may be lactose, corn starch, soybean oil, microcrystalline cellulose, or mannitol, and a lubricant may be magnesium stearate, talc, or a combination thereof. The carrier may be an excipient, a disintegrant, a binder, a lubricant, or a combination thereof. The excipient may be microcrystalline cellulose, lactose, low-substituted hydroxy cellulose, or a combination thereof. The disintegrant may be calcium carboxymethylcellulose, sodium starch glycolate, anhydrous calcium monohydrogen phosphate, or a combination thereof. The binder may be polyvinylpyrrolidone, low-substituted hydroxypropyl cellulose, hydroxypropyl cellulose, or a combination thereof. The lubricant may be magnesium stearate, silicon dioxide, talc, or a combination thereof.

The composition may be administered in various oral or parenteral dosage forms at the time of practical clinical administration. When formulated, diluents or excipients, such as fillers, extenders, binders, wetting agents, disintegrants, surfactants, etc. may be used in the preparation. Appropriate formulations known in the art are as disclosed in a literature (Remington's Pharmaceutical Science, latest edition, Mack Publishing Company, Easton Pa.).

Still another aspect provides a method of preventing or treating a disease, the method including administering an effective amount of the pharmaceutical composition according to an aspect to a subject in need thereof.

The “disease” is not limited to its type, as long as it is a disease that may be treated by gene editing. Therefore, the disease may be a disease caused by genetic mutation.

The disease may be a disease associated with a fusion gene. When Cas9-RNP bound to nanoparticles are two or more types, diseases associated with the fusion gene may be treated. For example, BCR-ABL fusion gene is a fusion gene found in chronic myelogenous leukemia, and FGFR3-TACC3 fusion gene is a fusion gene found in cervical cancer, etc. The fusion gene has similar characteristics to its original gene, but patients with the fusion gene show low sensitivity to traditional inhibitor drugs, and thus new alternatives such as multiple gene co-editing are needed. Since the nanoparticles according to one aspect have excellent Cas9-RNP loading and delivery capabilities, it is possible to treat diseases associated with fusion genes by targeting multiple genes. In one specific embodiment, the disease may be a cancer. The cancer may be selected from the group consisting of glioblastoma, lung adenocarcinoma, squamous cell lung carcinoma, head and neck squamous cell carcinoma, bladder cancer, cervical cancer, breast invasive ductal carcinoma, urothelial carcinoma, malignant glioma, and glioma, but is not limited thereto.

The “subject” refers to a subject in need of treatment of a disease. The subject may be a mammal, for example, human or non-human primates, mice, rats, dogs, cats, horses, cows, etc., but is not limited thereto.

The “administering” refers to an action for delivering a target object to a target. The administration dosage may be easily determined by those skilled in the art according to a patient's sex, age, body weight, health conditions, type of disease, severity, administration method, administration time, administration route, duration of treatment, and other factors well known in the medical field. The administration route and method are not particularly limited, and may follow any administration route and method, as long as the complex may reach a target site. Specifically, the complex may be administered through a variety of oral or parenteral routes, and non-limiting examples of the administration route include oral, rectal, topical, intravenous, intraperitoneal, intramuscular, intraarterial, transdermal, intranasal, or intraocular administration, etc.

The composition may be used alone or in combination with surgery, hormone therapy, chemotherapy, and methods of using biological response modifiers for the prevention or treatment of diseases.

In the present disclosure, the redundant contents are omitted in consideration of complexity of the present disclosure, and the terms not otherwise defined in the present disclosure have the meanings commonly used in the technical field to which the present disclosure pertains.

### BRIEF DESCRIPTION OF THE DRAWINGS

The above and other aspects, features, and advantages of certain embodiments of the disclosure will be more apparent from the following description taken in conjunction with the accompanying drawings, in which:

FIG. 1 shows an illustration of a mechanism of action of LPS-mediated CRISPR/Cas9 gene editing;

FIG. 2A shows a transmission electron microscopy (TEM) image of PS, Scale bar, 200 nm;

FIG. 2B shows a TEM image of LPS, Scale bar, 200 nm;

FIG. 2C shows BET surface area, pore-volume, mean pore size, and zeta potential of PS and LPS;

FIG. 2D shows characteristics of Cas9-RNP loaded on LPS;

FIG. 2E shows a Cas9-RNP release rate (%) of LPS over time;

FIG. 3 shows a gel image after dispersing Cas9-RNP@LPS and Cas9-RNP in PBS with protease, respectively;

FIG. 4 shows a graph of viability of HeLa cells according to concentrations of PS and LPS;

FIG. 5A shows independent fluorescence images corresponding to Cas9 (red) and LPS (green) in cytoplasm or nucleus at 48 hours after treatment, indicating delivery and release of Cas9-RNP from the LPS inside cells;

FIG. 5B shows fluorescence images of HeLa cells treated with Cas9-RNP@LPS for 48 hours;

FIG. 6 shows fluorescence images showing intracellular uptake of Cas9-RNP;

FIG. 7 shows rapid evaluation of model gene editing efficacy using quantification of GFP expression;

FIG. 8A shows a notable decrease in green fluorescence in cells treated with Cas9-RNP@LPS as compared to that in cells treated with Cas9-RNP/Lipo, Cas9-RNP@PS, Lipo, PS and LPS alone, wherein (+) control is a group treated with siRNA for transient gene knockdown;

FIG. 8B shows flow cytometry results showing a significant shift in cell population histograms correlated with fluorescence signal in Cas9-RNP@LPS-treated cells as compared to a control group;

FIG. 8C shows a bar graph presenting a percentage decrease in mean fluorescence intensity in cells treated with Cas9-RNP@LPS (59.0%), Cas9-RNP@PS (20.7%), and Cas9-RNP/Lipo (19.1%) relative to the untreated control, **p<0.01;

FIG. 9A shows fluorescence in controls;

FIG. 9B shows relative cell viability of cells treated with different materials;

FIG. 10A shows that the F-T fusion gene is encoded in chromosome 4;

FIG. 10B shows relative cell proliferation analyzed by MTS assay after treatment with FGFR3- or TACC3-targeting Cas9-RNP;

FIG. 10C shows relative caspase 3/7 activity;

FIG. 11A shows relative cell viability after treatment with Lipo alone;

FIG. 11B shows that 8×Lipo allowed the highest gene editing efficiency relative to that of Cas9-RNP@LPS;

FIG. 11C shows the results of 11A and 11B taken together, wherein 4×Lipo was applied for the subsequent study, considering efficacy and safety;

FIG. 12A shows RT-PCR results;

FIG. 12B shows Western blotting results, *p<0.05, **p<0.01;

FIG. 12C shows that knockout of FGFR3 and TACC3 inhibited activations of both ERK and Akt;

FIG. 12D and FIG. 12E show analysis of the mutation frequency of several potential targets of Cas9-RNP by next-generation sequencing;

FIG. 13A shows real-time whole-body images after intravenous injection with Cas9-RNP or Cas9-RNP@LPS;

FIG. 13B shows TEM images;

FIG. 14A shows fluorescence signals in liver, lung, spleen, kidney, heart, and tumor after intravenous injection with Cas9-RNP or Cas9-RNP@LPS;

FIG. 14B shows fluorescence signals in liver, lung, spleen, kidney, heart, and tumor after intravenous injection with Cas9-RNP or Cas9-RNP@LPS;

FIG. 14C shows fluorescence signals in mouse whole bodies after intravenous injection with Cas9-RNP or Cas9-RNP@LPS;

FIGS. 15A-15B show anti-tumor activity of Cas9-RNP by genome editing of multiple genes (FGFR3 and TACC3) in a human cancer xenograft mice model, **p<0.01, ***p<0.001;

FIG. 16 shows hematoxylin and eosin staining results showing intensive apoptosis only in tumors treated with Cas9-RNP@LPS;

FIG. 17 shows histological images showing viability of major organs after injection with Cas9-RNP@LPS;

FIG. 18A shows results of a Western blot comparison of a targeting F-T fusion gene and multiple genes having high similarity to the F-T fusion gene;

FIG. 18B shows results of a relative cell viability comparison of a targeting F-T fusion gene and multiple genes having high similarity to the F-T fusion gene; and

FIG. 18C shows mutation rates according to multiple gene targeting.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects of the present description. As used herein, the term “and/or” includes any and all combinations of one or more of the associated listed items. Expressions such as “at least one of,” when preceding a list of elements, modify the entire list of elements and do not modify the individual elements of the list.

Hereinafter, the present disclosure will be described in more detail with reference to exemplary embodiments. However, these exemplary embodiments are only for illustrating the present disclosure, and the scope of the present disclosure is not limited to these exemplary embodiments.

Preparation of Materials

Cetyl trimethyl ammonium bromide (CTAB) was purchased from Acros (New Jersey, USA).

3-Aminopropyltriethoxysilane (APTES), tetramethyl orthosilicate (TMOS), toluene, dimethylsulfoxide (DMSO), and mesitylene (trimethyl benzene, TMB) were purchased from Sigma-Aldrich (St. Louis, Mo., USA).

10× Phosphate buffered saline (PBS), Dulbecco's modified eagle's medium (DMEM), fetal bovine serum (FBS), and penicillin and streptomycin (P/S, 100×) were purchased from WELGENE, Korea.

A CCK-8 assay kit was purchased from Dojindo Molecular Technologies, Inc, USA. Lipofectamine™ CRISPRMAX™ Cas9 transfection reagent was purchased from ThermoFisher Scientific, USA.

Trizol for total RNA isolation, and Superscript II reverse transcriptase were purchased from Life technologies, USA.

Primers and siRNAs were purchased from Bioneer, Korea.

Cas9 protein and sgRNAs were purchased from ToolGen, Inc. Korea.

### COMPARATIVE EXAMPLES AND EXAMPLES: PREPARATION OF CAS9-RNP DELIVERY PLATFORM

### Comparative Example 1: Preparation of Silica Nanoparticle (PS) of Porous Structure

To prepare monodisperse PS, a TMOS solution was added by using a tube-connected syringe-pump at 0.5 mL/h into 4 g of CTAB dissolved in a basic solution (1 M NaOH), and stirred for 6 hours and left overnight without stirring. The synthesized product was centrifuged and washed with ethanol and water.

For surface modification, PS was suspended in APTES solution, and the suspension was refluxed at 120° C. overnight, and washed with ethanol and water.

### Example 1: Preparation of Silica Nanoparticle (LPS) of Porous Structure Having Large Pore Size

Silica nanoparticles of a porous structure having a pore size of 20 nm or more were prepared.

In detail, a TMOS solution was added by using a tube-connected syringe-pump at 0.5 mL/h into 4 g of CTAB dissolved in a basic solution (1 M NaOH), and stirred for 6 hours and left overnight without stirring. The synthesized product was centrifuged and washed with ethanol and water to obtain monodisperse PS.

To obtain LPS, the obtained PS was dissolved in a mixture of TMB:EtOH:DW (1:1:1, v/v) and allowed to react in an autoclave at 160° C. for 48 hours. The resulting powder was washed with ethanol and water. To remove the CTAB, 12 M of HCl was added to LPS in ethanol, and refluxed at 120° C. overnight.

For surface modification, LPS was suspended in an APTES solution, and the suspension was refluxed at 120° C. overnight, and washed with ethanol and water.

Experimental Methods

(1) Characterization of PS and LPS

The pore size, surface area, and pore volume of PS and LPS were analyzed through nitrogen sorption experiments. Nitrogen sorption isotherms were obtained using a NOVA surface area analyzer (Nova 2200e, Quantachrome instrument, USA). Before the measurements, the sample was degassed for 12 hours at 573 K. The morphological study was carried out using transmission electron microscopy (TEM, JEM1010, JEOL, Japan) and zeta potential was measured by zetasizer NS90 (Malvern, UK).

(2) Cell Culture

HeLa cell line was obtained from American Tissue Culture Collection (ATCC). The cells were incubated in DMEM (LM001-05, Welgene) containing 10% Fetal bovine serum (FBS, S001-01, Welgene) and 1% penicillin-streptomycin (PS, LS202-02, Welgene) under conditions of 5% CO₂ at 37° C. The HeLa cells were isolated from the cell culture plate with a 0.05% trypsin (LS015-01, Welgene). 10 μL of the cell solution were stained with a same amount of tryptophan blue (0.4%), spread on an EVE™ cell counter slide (EVS-050) and counted with an EVE™ automated cell Counter (NanoEnTek).

(3) Cell Proliferation Assay

To verify biocompatibility of a carrier system, a cell proliferation test was conducted with a CellTiter96 aqueous one solution cell proliferation assay (MTS, G5440) purchased from Promega. A 96-well plate was used for the cell culture, and the cells were seeded at the number of 10,000 per each well. After the cells were washed with PBS twice, the same amount of PBS was injected with an increasing concentration of particles. The MTS assay was then performed according to the manufacturer's instructions. After incubation for 30 minutes at 37° C., fluorescence intensity was measured with an Infinite F200 pro (TECAN, USA).

(4) Flow Cytometry

To measure the GFP knockout efficiency, GFP-HeLa cells (3×10⁴ cells/well, 24-well plate) were treated with a complex in serum-free media for 4 hours. Then, the cell medium was replaced by a serum-containing fresh medium and incubated for 44 hours. After washing with 1×PBS, the cells were collected after treatment with trypsin-EDTA for 3 minutes, and then 10% FBS was added to the collected cells. After centrifugation (1,200 rpm, 3 minutes), the cells were washed with PBS, and the fluorescence of the cells was measured by a flow cytofluorometer, FACS Canto (Beckton Dickinson, USA).

(5) Cellular Imaging

To verify whether the LPS penetrates into the cells and the inner contents of the LPS spread out without forming lysosome, FITC-labelled LPS, and Cy5-labelled Cas9 protein were prepared, and then HeLa cells (2×10⁴ cells/well, 24-well plate) were treated with the complex in a serum-free medium for 4 hours. The cell medium was replaced by a serum-containing fresh medium and incubated further 24 hours and 48 hours for time-dependent monitoring. At a certain time-point, the cells were fixed with 4% paraformaldehyde (HP2031, biosesang, Korea) followed by DAPI staining (H-1200, Vector Laboratories). Fluorescence images were monitored with a confocal microscope (Zeiss) and analyzed with the Image J software.

(6) RT-PCR

Total RNA was isolated from each sample with TRIZOL reagent, followed by cDNA synthesis aided by Superscript™ reverse transcriptase according to the instruction manual. For target gene amplification, each primer was designed in consideration of GC content less than 50% and overlapping between two exons of the target genes. By gel electrophoresis on 1 agarose, the PCR products were separated and the bands were monitored.

(7) Western Blot

The samples were lysed with RIPA buffer (AKR-190, Koma) containing 100× protease inhibitor cocktail and incubated for 1 hour on ice. Thereafter, the samples were centrifuged at 12,000 rpm and 37° C. for 15 minutes. The upper part of the solution was transferred to a 1.5 mL ep-tube and stored at −80° C. For protein quantification, a Pierce™ BCA assay kit (#23225, ThermoFisher Scientific) was used and the protocols were followed by the manufacturer's instruction. The same amount of protein was diluted in water respectively containing 5× loading buffer (S2002, Biosesang) and loaded on SDS-PAGE gel (Biorad). By electrophoresis in Tris-Glycine SDS buffer at 80 V for 120 minutes, the proteins were completely separated. The proteins on SDS-PAGE gel were transferred to a nitrocellulose membrane of 0.45 μm (#1620145, biorad) in 1× Tris-Glycine Native Buffer (HT2028, Gendepot) with 10% methanol at 50 V, 4° C. for 120 minutes under stirring condition. The membrane was blocked with 3% BSA to inhibit non-specific binding and immersed overnight at 4° C. in primary antibodies; GAPDH (ab9485, Abcam, 1:2500), TACC3 (sc-376883, Santa cruz, 1:1000), and FGFR3 (90313-T48, Sino, 1:1000). Horse-Radish-Peroxidase (HRP)-conjugated antibodies, reactive with rabbit (ab6721, Abcam, 1:5000) and mouse (ab6789, abcam, 1:5000) were used as a secondary antibody and the membrane was incubated for 1 hour. After reaction with a detection agent EzWestLumi plus (WSE-7120L, ATTO Corporation), protein bands were monitored by ImageQuant LAS 4000 mini.

(8) Genomic DNA Sequencing

Genomic DNA was collected from HeLa cells using AccuPrep® Genomic DNA Extraction Kit (Bioneer, Korea). The extracted genomic DNAs were amplified using SUN-PCR blend (SUN GENETICS) for sequencing library generation. The libraries were sequenced using a Mini-Seq instrument (Illumina). The results of Mini-seq were analyzed using Cas-Analyzer web tool (http://www.rgenome.net/be-analyzer/).

(9) Mouse Study

All animal experiments were carried out in compliance with the Institutional Animal Care and Use Committees (IACUC) of Korea Institute of Science and Technology. For the real-time in vivo monitoring, Cy5-Cas9 and Cy7-LPS were prepared. Mice were inoculated subcutaneously with HeLa cells in the right flank and randomly divided into three groups. After two-week incubation, each group was intravenously administered with Cas9-RNP@LPS. Bright-field and fluorescence images from the mice were periodically obtained using in vivo fluorescence microscopy. All acquisition image was operated for pseudo-coloring with fluorescent intensity.

To investigate the anti-tumor therapeutic effect, xenograft mouse model was prepared by implanting 1×10⁶ of HeLa cancer cells to Balb/c nude male mouse with subcutaneous injection every three days after one-week breeding. Anti-tumor efficacy was evaluated by measuring the change of tumor volume before/after injection with free LPS, free TACC3/Lipo, FGFR3/Lipo, TACC@LPS, FGFR3@LPS (6 mg mL-1), and 1×PBS for 15 days (n=3). The tumor volumes were calculated using the following equation: Tumor volume=Length×(width)²×½, wherein the length and width are the longest and shortest diameters (mm) of the tumor, respectively. The tumor volumes were calculated relative to the initial volumes (100 mm³).

(10) Histological Analysis

Tumor tissues were extracted from euthanized mice and incubated in a 4% paraformaldehyde solution at 4° C. for fixation. The tissues were embedded in paraffin blocks and sliced into 5 μm sections with Leica Microtome. The tissue sections were histologically visualized. The paraffin sections were soaked into Xylene three times for deparaffinization and hydrated with a series of decreasing ethyl alcohol. For hematoxylin and eosin staining, the sections were soaked into Hematoxylin solution and subsequently into Eosin solution. The sections were sandwiched with mounting buffer and coverslip, and observed with an optical microscopy (Leica). For immunofluorescence staining, primary and secondary antibodies were used to specifically monitor a targeting protein. First, to increase the specificity and reactivity of the antigen, the deparaffinized and hydrated sections were blocked with 0.3% H₂O₂ in methanol for 30 min and soaked in citrate buffer for 20 min at 95° C. To prevent non-specific binding, the sections were blocked with 3% BSA for 1 hour in a static condition. Thereafter, the sections were incubated overnight at 4° C. with primary antibodies; anti-TACC3 (202326-T10, sino, 1:500), anti-FGFR3 (90313-T48, sino, 1:500). A secondary antibody Alexa Fluor™ 488 goat anti-rabbit IgG (H+L) (A-11008, Invitrogen, 1:5000) was used and treated for 1 hour. The sections were mounted with a Vectashield mounting medium with DAPI (H-1200, Vector Laboratories) and observed using an LSM 700 confocal microscope (Zeiss).

(11) Statistics and Data Analysis

All statistical analyses were performed with unpaired t-test, GraphPad Prism 8 software. p<0.05 was considered significant (*p<0.05, **p<0.01, ***p<0.001 and ****p<0.0001); n.s., not significant). All the results are given as means±standard deviation.

(12) Design of sgRNA

sgRNAs of the CRISPR/Ca9 system were designed as in the sequences of Table 1 below. In the following experiments, sgRNAs of SEQ ID NO: 1, SEQ ID NO: 3, SEQ ID NO: 6, and SEQ ID NO: 9 were used.

TABLE 1

| Target | | |
|---|---|---|
| gene | sgRNA sequence | SEQ ID NO: |
| GFP | 5′ CCAGGGCACGGGCAGCTTGCCGG 3′ | 1 |
| | 5′ GGCGAGGGCGATGCCACCTACGG 3′ | 2 |
| FGFR3 | 5′ ACGCGGCCAAGGGTAACCTGCGG 3′ | 3 |
| | 5′ GAAGGAGTAGTCCAGGCCCGGGG 3′ | 4 |
| | 5′ AGGTGTCGAAGGAGTAGTCCAGG 3′ | 5 |
| TACC3 | 5′ CGGAGTCTCCGCTTTGTGCTGGG 3′ | 6 |
| | 5′ AGACCCTAGAAGACCCTTGCAGG 3′ | 7 |
| | 5′ GCCGAGGAGGAATGCCGGCACGG 3′ | 8 |
| F-T | 5′ TTCGAGCAGTACTCCCCGGGTGG 3′ | 9 |

Experimental Results

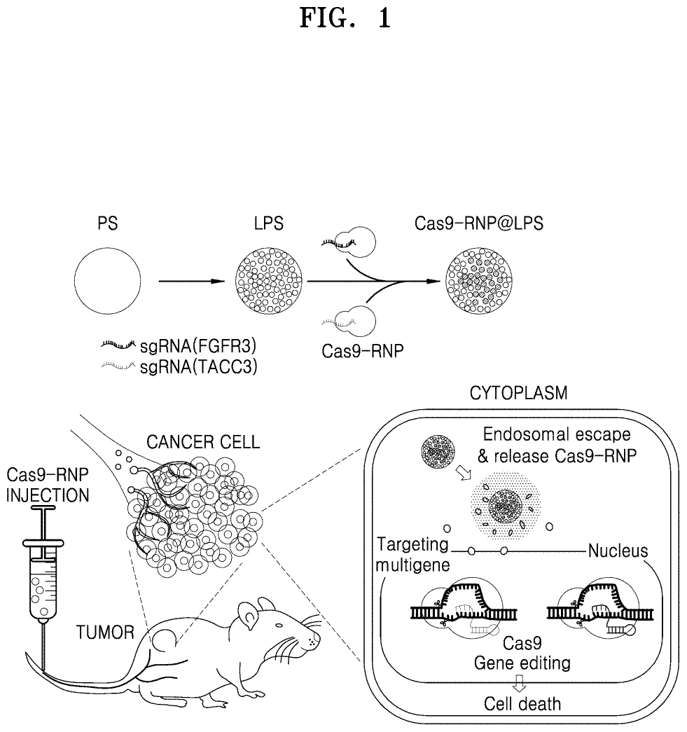

FIG. 1 shows an illustration of a mechanism of action of LPS-mediated CRISPR/Cas9 gene editing.

The present inventors synthesized LPS possessing a large pore size of 20 nm or more with high surface area and cavity. The LPS was subsequently functionalized with positively charged groups capable of being loaded with Cas9-RNP via electrostatic interactions. The expanded pore and high surface area of the LPS provides enough inner space to enable a high loading capacity of large-sized protein and protect Cas9-RNP from the proteolytic environment.

Importantly, the synthesis process of Cas9-RNP@LPS complex was achieved at mild conditions such as gentle mixing and reaction at room temperature, which contribute to maintaining the enzymatic activity of Cas9 protein. The LPS outer surface property of presenting amine functional groups may contribute to an excellent buffering capacity, and may also enhance the escape probability from the endocytic pathway. In addition, the inner surface functionality of LPS and anionic characteristics of Cas9-RNP may be affected by the change of pH and ionic strength, thus enabled the release of Cas9-RNP from the LPS through the reduction of electrostatic interaction and hydrogen bonding interaction between LPS and Cas9-RNP. Therefore, released Cas9-RNP may enter the nucleus to initiate site-specific gene editing. The efficacy of genetic engineering was assessed by monitoring tumor suppression in vivo mouse model and NGS analysis of a mutation rate. Detailed experimental processes confirming the above are shown below.

### Experimental Example 1: Characterization of PS and LPS

(1) Characteristics of PS and LPS

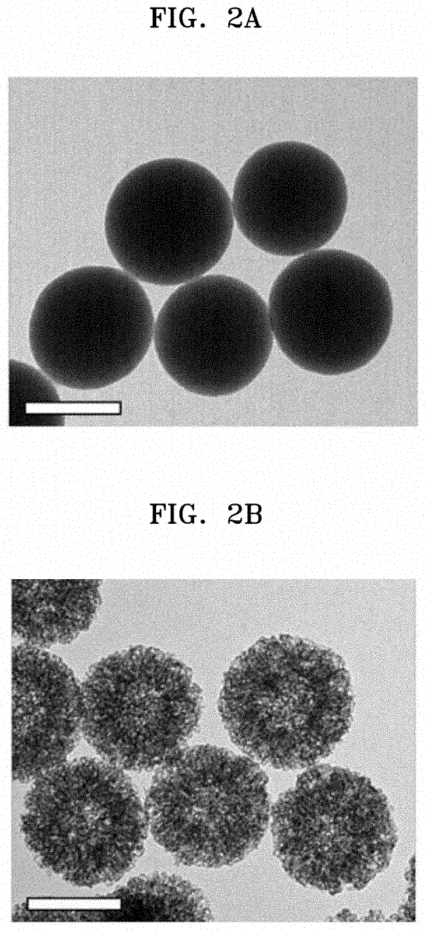

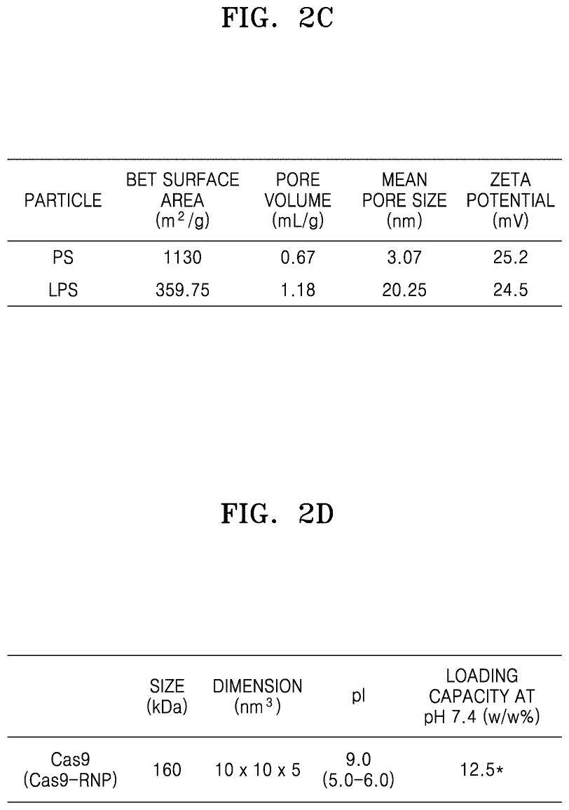

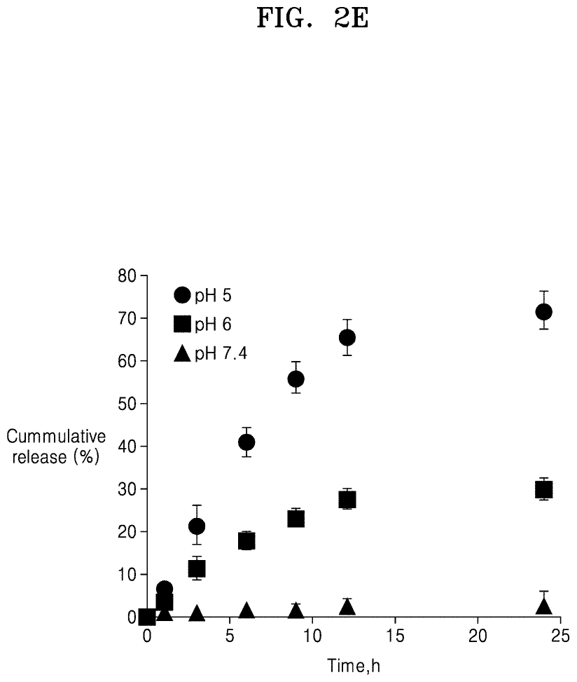

FIG. 2A to FIG. 2E show results of characterization of PS and LPS.

As shown in FIGS. 2A and 2B, the transmission electron microscopy (TEM) images showed porous 200-nm sized particle morphology of PS and LPS.

As shown in FIG. 2C, the results for nitrogen sorption and zeta potential analysis showed that the prepared LPS possessed expanded pore size (˜21 nm) and positive surface charge (24.5 mV), which may enable loading of Cas9-RNP inside the pore.

Next, Cas9-RNP-loaded LPS (Cas9-RNP@LPS) was obtained by simple mixing of 8 μg of Cas9-RNP and 60 μg of LPS prepared in Example 1 in phosphate-buffered saline (PBS) for 30 minutes. The Cas9-RNP loading capacity of LPS was evaluated using a bicinchoninic acid (BCA) assay.

As shown in FIG. 2D, the Cas9-RNP loading capacity of LPS was 12.5% (w/w). The LPS according to the present embodiment is larger than that of previous reported nanoparticle-based delivery systems (Adv. Funct. Mater. 2017; 27(46): 1703036; J. Am. Chem. Soc. 2018; 140(1):143-136) having the loading capacity of 6.3% and 1.2%, respectively.

Next, the present inventors characterized the release of Cas9-RNP from LPS under the various pH conditions by absorption spectroscopy with monitoring for 24 hours.

As shown in FIG. 2E, under physiological conditions at pH 7.4, less than 5% of Cas9-RNP was released in 12 hours, while 30% and 70% were released at pH 6 and pH 5, respectively. This indicates that the release of Cas9-RNP was more favorable under the acidic environment which is close to the endosomal conditions. Furthermore, this sustained-release phenomenon of LPS is beneficial for maintaining Cas9 protein activity with high stability for a long time in vivo environment, and is superior to that of other reported delivery systems.

(2) Verification of Stability of Cas9-RNP Inside LPS and Protective Effect of LPS Under Proteolytic Environment

To evaluate stability of Cas9-RNP inside LPS and a protective effect of LPS under physiological conditions, Cas9-RNP@LPS and Cas9-RNP were dispersed in PBS with protease.

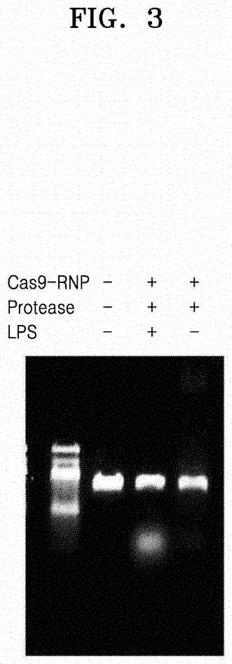

FIG. 3 shows a gel image after dispersing Cas9-RNP@LPS and Cas9-RNP in PBS with protease, respectively.

As shown in FIG. 3, in vitro cleavage activity was observed in the Cas9-RNP@LPS-treated group, while no significant cleaved band of the target DNA was observed in the Cas9-RNP alone-treated group. This result indicates that LPS exhibited the superb Cas9 protecting ability in the proteolytic environment and the released Cas9 protein was still active.

(3) Test of Biocompatibility of PS and LPS

Next, the present inventors tested the biocompatibility of materials using HeLa to increase the concentration of PS and LPS.

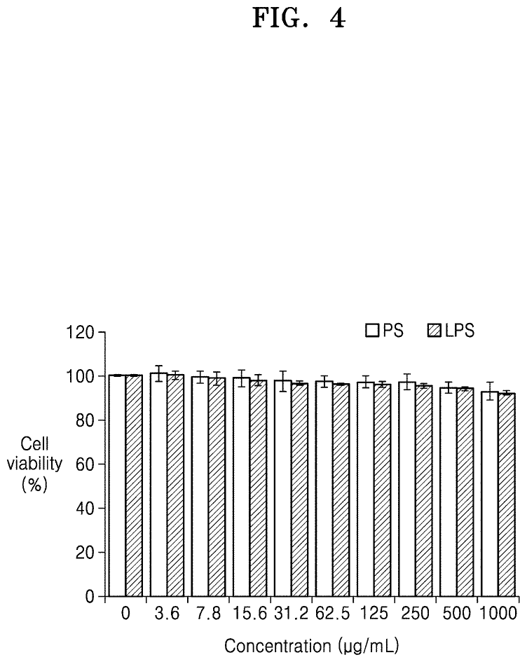

FIG. 4 shows a graph of viability of HeLa cells according to concentrations of PS and LPS.

As shown in FIG. 4, it was found that more than 90% of the cells were viable under all concentrations of PS and LPS. Such superb biocompatibility of Cas9-RNP@LPS may guarantee the potential of LPS platform for gene editing platform.

### Experimental Example 2: Evaluation of Intracellular Uptake of Cas9-RNP

To evaluate intracellular uptake of Cas9-RNP@LPS, Cas9 protein and LPS were labeled with Cy5 (Cy5-Cas9) and FAM (FAM-LPS), respectively before formation of Cas9-RNP@LPS complex. After the treatment of HeLa cells with Cas9-RNP@LPS complex, the cells were visualized using fluorescence microscopy.

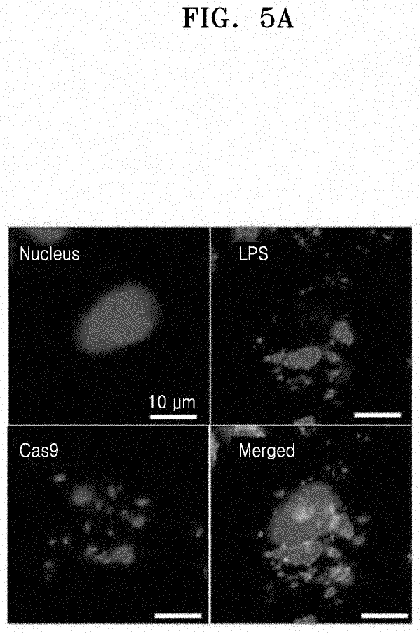

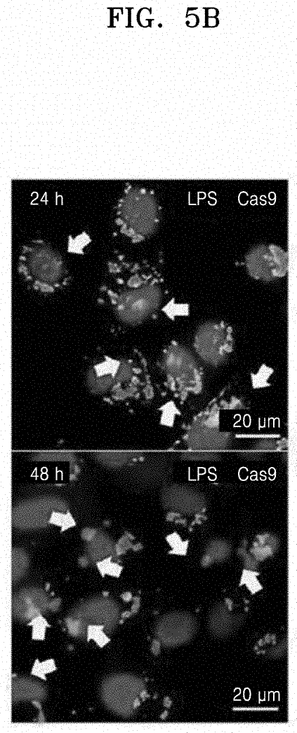

FIG. 5A shows independent fluorescence images corresponding to Cas9 (red) and LPS (green) in cytoplasm or nucleus at 48 hours after treatment. This indicates delivery and release of Cas9-RNP from the LPS inside cells.

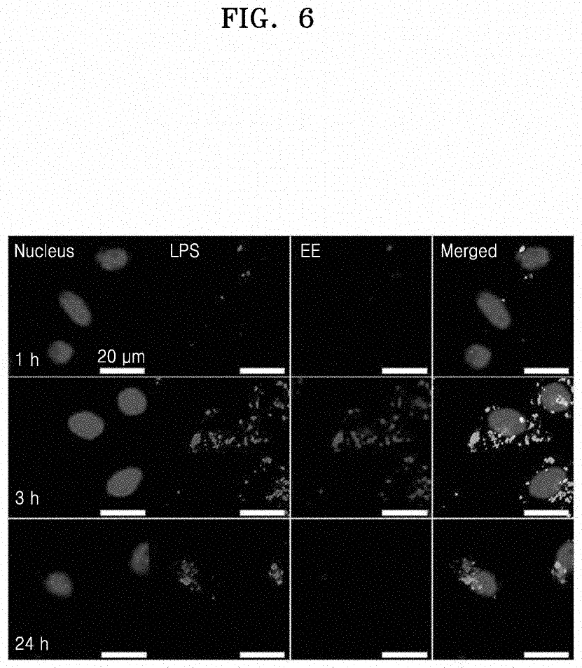

FIG. 6 shows fluorescence images showing intracellular uptake of Cas9-RNP.

As shown in FIG. 5, fluorescence corresponding to Cas9 and LPS were observed in cytoplasm or nucleus independently after 24 hours of treatment. This result indicates the successful delivery and release of Cas9-RNP from the LPS inside the cells.

Further, as shown in FIG. 6, the green fluorescence of Cas9-RNP@LPS complex was separated from the yellow color which was the merged color with endosome, and red fluorescence gradually disappeared. This result suggests the release of the Cas9-RNP@LPS complex to the cytoplasm over time. This result implicated that LPS showed the ability to escape the endocytic pathway, owing to the buffering capacity of surface functional groups.

### Experimental Example 3: Evaluation of In Vitro Gene Editing Efficacy of Cas9-RNP

LPS-mediated gene editing efficacy in living cells was evaluated. To quantitatively measure the knockout of gene expression, a green fluorescence protein (GFP) was preferentially selected as a model gene to be able to rapidly visualize the mutation rate in cells. GFP-expressing HeLa cells were treated with Cas9-RNP@LPS (1.25 μg of Cas9) in a serum-free medium in a 24-well plate for 4 hours, suitable conditions for in vitro and ex vivo applications, followed by replacement with a fresh serum-containing medium. A common regent, lipofectamine (Lipo) was used as a control. Relative GFP expression levels were estimated by observing fluorescent images of the cells using a fluorescence microscope and flow cytometry.

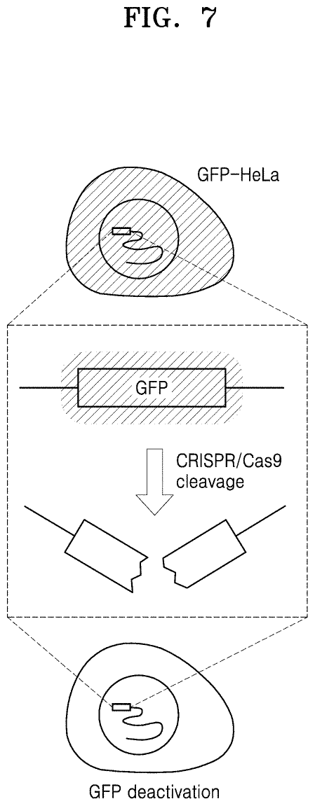

FIG. 7 shows rapid evaluation of model gene editing efficacy using quantification of GFP expression.

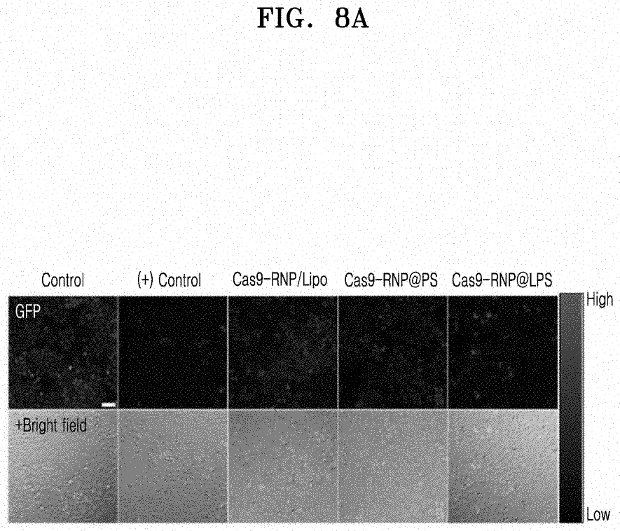

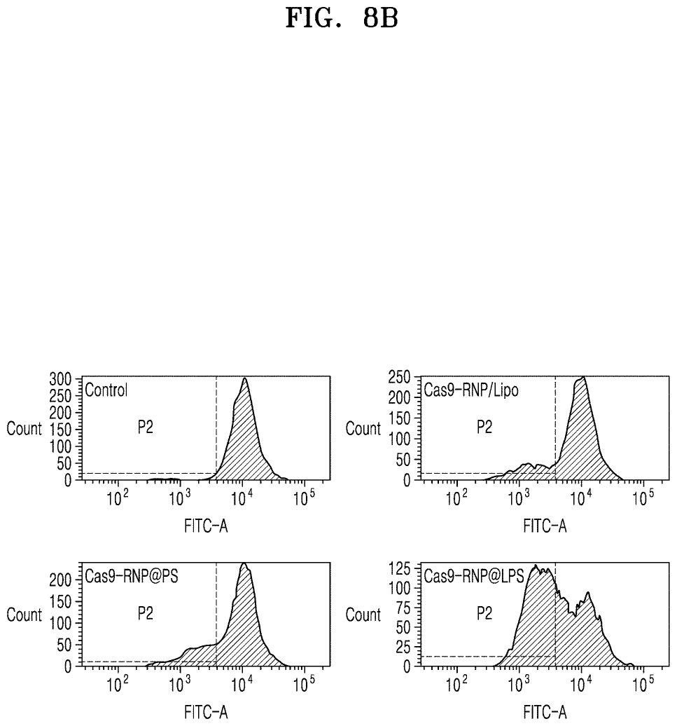

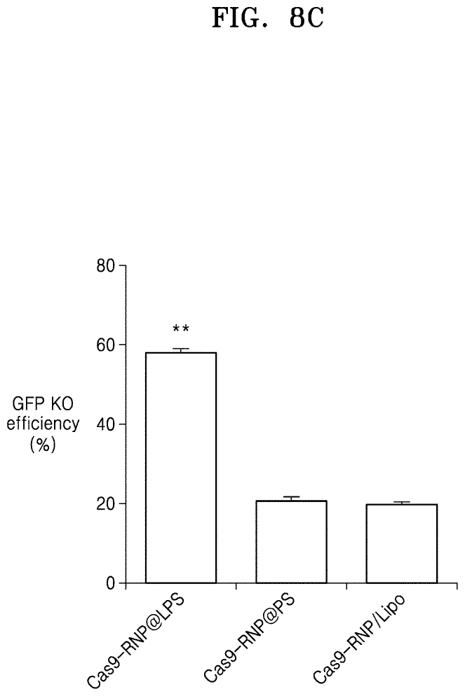

FIG. 8A shows a notable decrease in green fluorescence in cells treated with Cas9-RNP@LPS as compared to that in cells treated with Cas9-RNP/Lipo, Cas9-RNP@PS, Lipo, PS and LPS alone. (+) Control is a group treated with siRNA for transient gene knockdown.

FIG. 8B shows flow cytometry results showing a significant shift in cell population histograms correlated with fluorescence signal in Cas9-RNP@LPS-treated cells as compared to a control group.

FIG. 8C shows a bar graph presenting a percentage decrease in mean fluorescence intensity in cells treated with Cas9-RNP@LPS (59.0%), Cas9-RNP@PS (20.7%), and Cas9-RNP/Lipo (19.1%) relative to the untreated control.

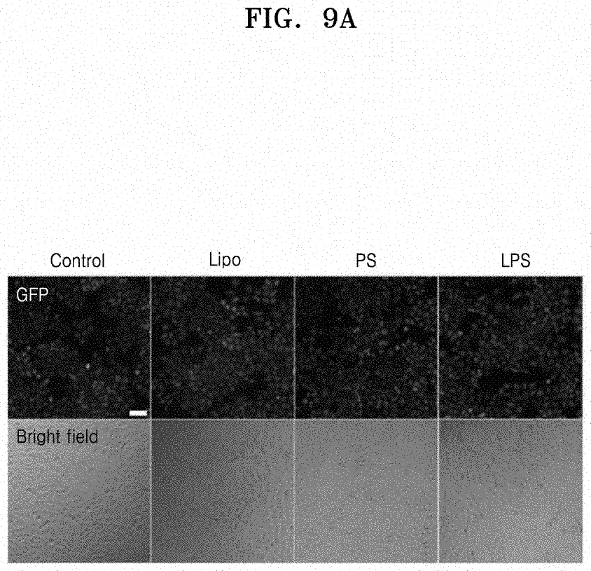

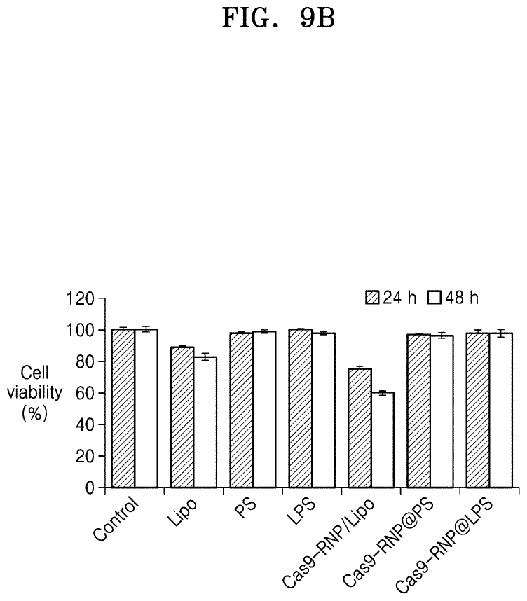

FIG. 9A and FIG. 9B show results of rapid evaluation of model gene editing efficacy using quantification of GFP expression and cell viability.

As shown in FIGS. 8A and 9A, a notable decrease in green fluorescence was observed in cells treated with Cas9-RNP@LPS as compared to the cells treated with Cas9-RNP/Lipo, Cas9-RNP@PS, Lipo, PS, LPS alone.

As shown in FIG. 8B, a significant shift was observed in the cell population histogram correlated with the fluorescence signal in Cas9-RNP@LPS-treated cells as compared to control groups.

As shown in FIG. 8C, the mean green fluorescence intensity also significantly decreased in the cells treated with Cas9-RNP@LPS down to 59.0% relative to the untreated control, as compared to those observed with Cas9-RNP@PS (20.7%) and Cas9-RNP/Lipo (19.1).

As shown in FIG. 9B, LPS-mediated Cas9-RNP delivery showed higher gene knockout than Lipo as well as higher cell viability (99.5%) as compared to Cas9-RNP/Lipo (59.5%) and Lipo (81.3%) alone for 48 hours. This result suggests that LPS is an efficient Cas9-RNP delivery vehicle with low toxicity.

Based on the preliminary result, the present inventors investigated the gene editing efficacy by targeting multiple genes, namely, FGFR3 and TACC3, which has high similarity to the F-T fusion gene.

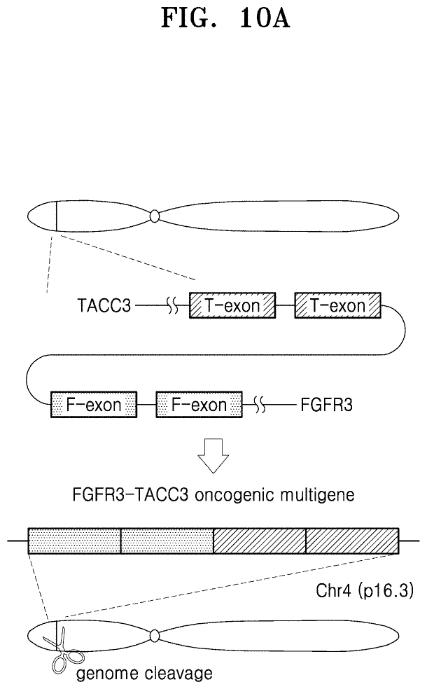

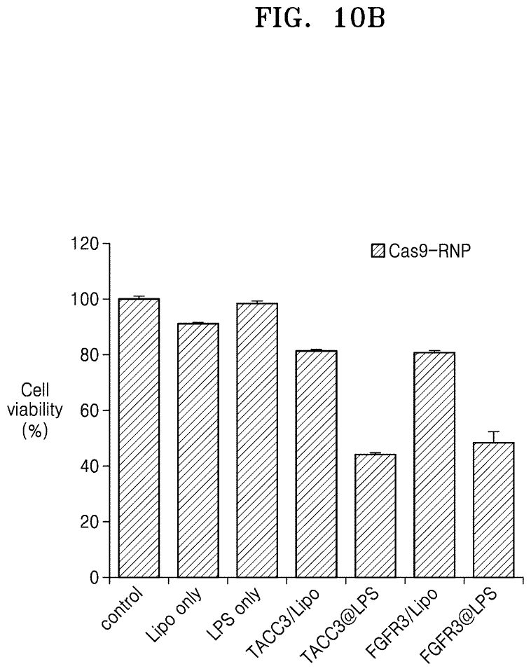

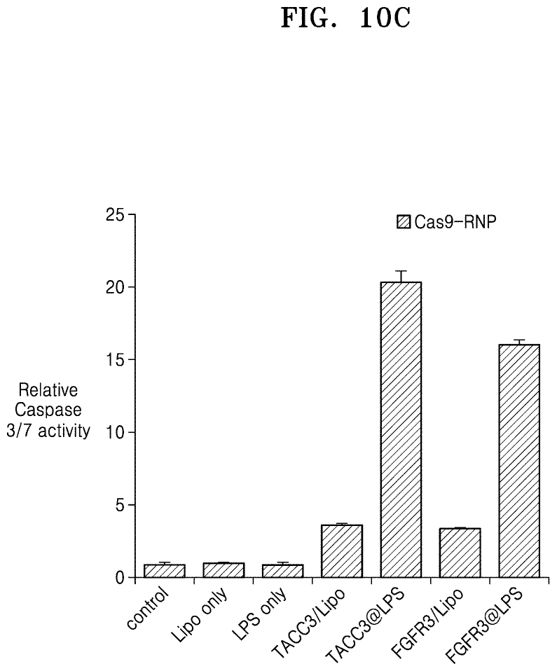

FIG. 10A to FIG. 10C show targeting of multiple genes that have high similarity to the F-T fusion gene.

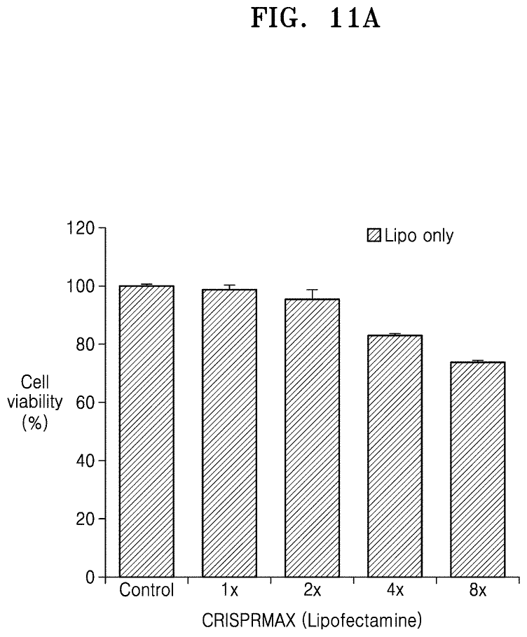

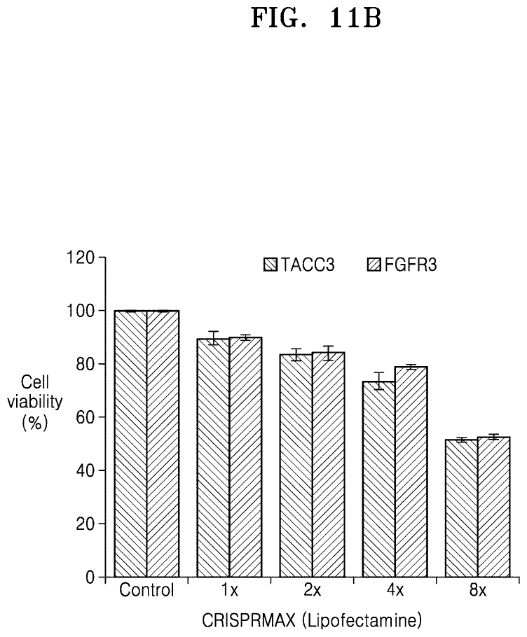

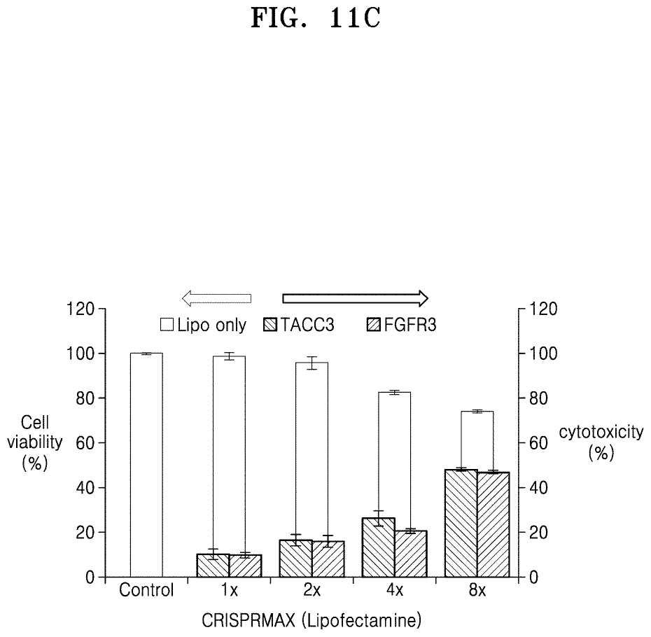

FIG. 11A to FIG. 11C show results of systemic optimization of Lipo-mediated Cas9-RNP delivery.

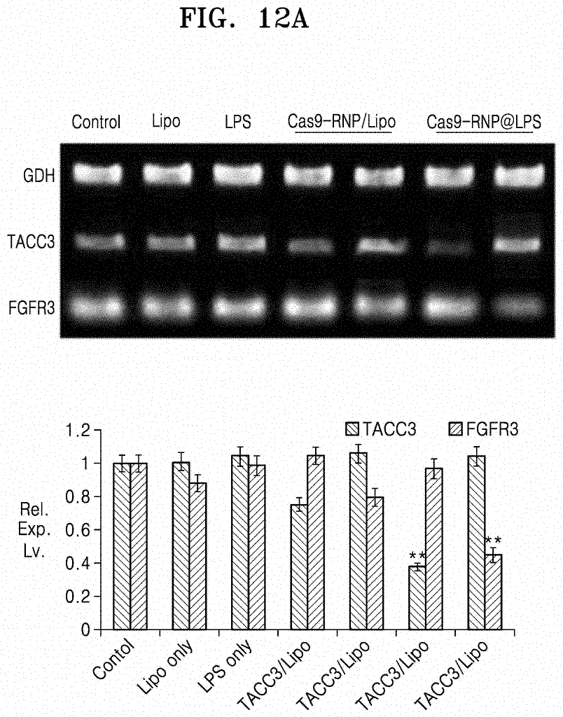

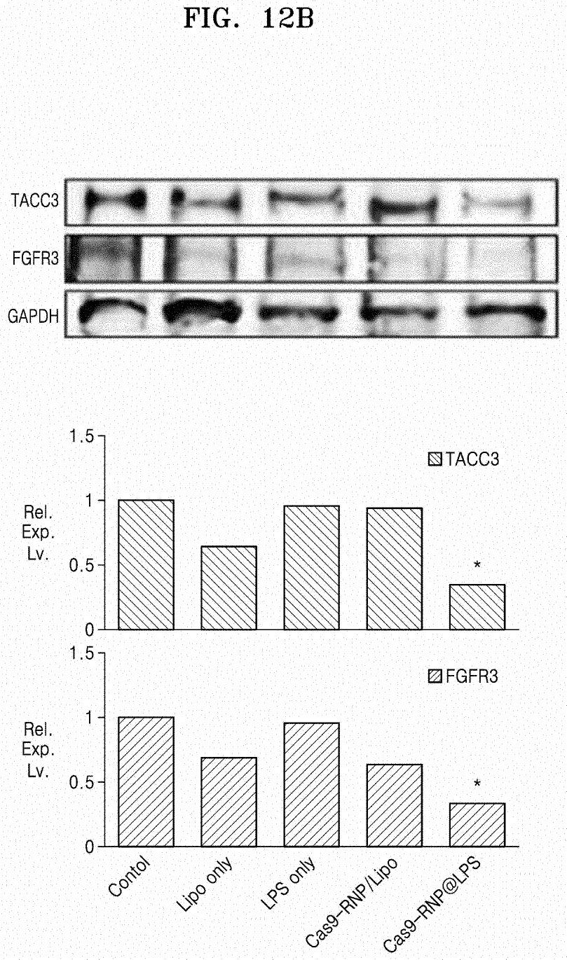

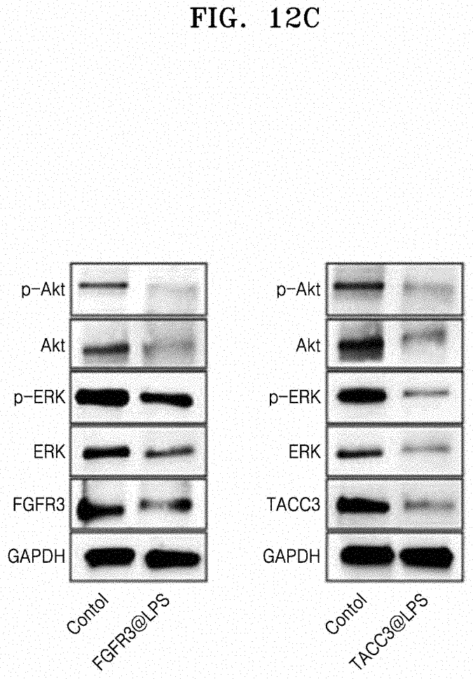

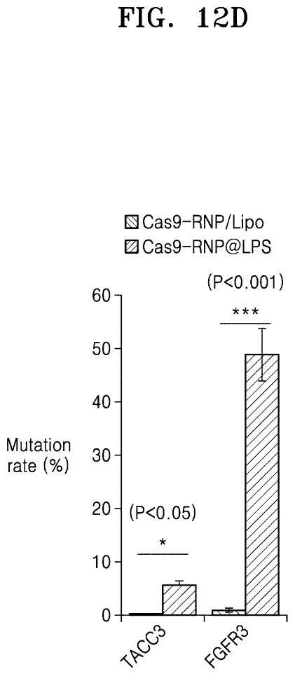

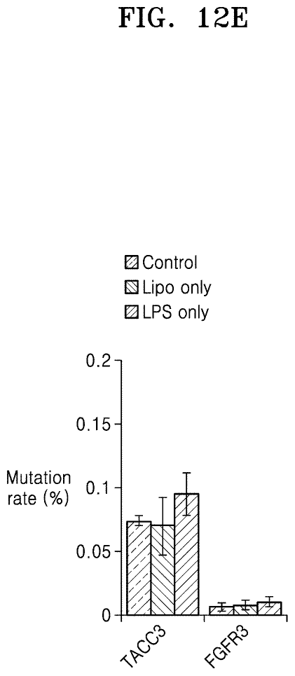

FIG. 12 shows results of evaluating efficacy of knocking out multiple genes in vitro.

As shown in FIG. 10A, both FAFR3 and TACC3 are encoded on chromosome 4 and show a low expression level at different cellular locations in normal cells. However, when each protein is overexpressed by gene fusion, cell proliferation is activated, leading to cancer.

Therefore, by cleaving multiple genes using the Cas9-RNP@LPS complex, a therapeutic effect may be expected via blocking of proliferation pathways and inducing apoptosis, leading to cell death.

As shown in FIG. 11, for precise comparison of Cas9-RNP delivery systems, Lipo-mediated trials were systemically optimized, wherein 8×Lipo allowed the highest gene editing efficiency, but with slight toxicities. Therefore, considering both efficacy and safety, 4×Lipo was applied for subsequent study.

The relative cell proliferation was analyzed after the treatment of cells with FGFR3- or TACC3-targeting Cas9-RNP by MTS assay.

As shown in FIGS. 10B and 10C, viability of cells treated with the Lipo-mediated Cas9-RNP complex remained over 80% for both FGFR3 and TACC3, whereas the Cas9-RNP@LPS-treated cells showed below 45% viability. The relative caspase 3/7 activity of the LPS-mediated RNP-treated group was five times higher than that of the Lipo-mediated RNP-treated group.

The efficacy was further evaluated at mRNA and protein levels, via RT-PCR and western blot, respectively, at 48 hours after treatment of HeLa cells.

As shown in FIGS. 12A and 12B, as expected, targeting of FGFR3 and TACC3 resulted in a decrease in gene expression up to 45% and 40%, and to less than 20% of the protein expression, while treatment with vehicle alone, or untreated controls did not result in gene editing.

Since the F-T fusion gene is involved in the activation of oncogenic pathways such as MAPK/ERK and PI3K/Akt signaling, the present inventors probed the effect of multiple gene editing on these signal transduction mechanisms.

As shown in FIG. 12C, knockout of FGFR3 and TACC3 inhibited activations of both ERK and Akt, which decreased the protein levels of phosphorylated ERK (p-ERK) and phosphorylated Akt (p-Akt). Furthermore, this indicated that knockout of multiple genes may suppress cervical cancer growth in vitro and in vivo via inhibition of both ERK and Akt signaling pathways.

Next, the present inventors analyzed the mutation frequency of the potential targets of Cas9-RNP, including the multiple genes, by NGS analysis.

As shown in FIGS. 12D and 12E, it was found that mutation rates were over 48.9% and 5.46% at FGFR and TACC3 loci by LPS-mediated Cas9-RNP gene editing, which showed over 50-fold enhancement as compared to that by Lipo-mediated editing. However, no significant mutation rate (below 0.1%) was observed in LPS alone, Lipo alone, and control groups. These results collectively showed the efficient multiple gene editing capability as well as high specificity of the Cas9-RNP delivery platform according to the present embodiment.

### Experimental Example 4: In Vivo Evaluation of Cas9-RNP@LPS-Mediated Gene Therapy

(1) Verification of Tumor-Specific Intracellular Delivery of Cas9-RNP

To test the efficacy of Cas9-RNP@LPS in vivo, the present inventors first experimentally monitored its distribution following systemic administration in a xenograft mouse model bearing HeLa cells. Cas9 protein and LPS were labeled with Cy5 (Cy5-Cas9) and Cy7 (Cy7-LPS), respectively, before formation of the Cas9-RNP@LPS complex. Tumor-bearing mice were prepared by subcutaneously injecting a suspension of HeLa cells (6×10⁶ cells) in 100 μL of sterilized PBS into Balb/c male nude mice (5-week-old). Each fluorescence was monitored by whole-body imaging after intravenous injection with the complex, and then major organs were excised from the mice.

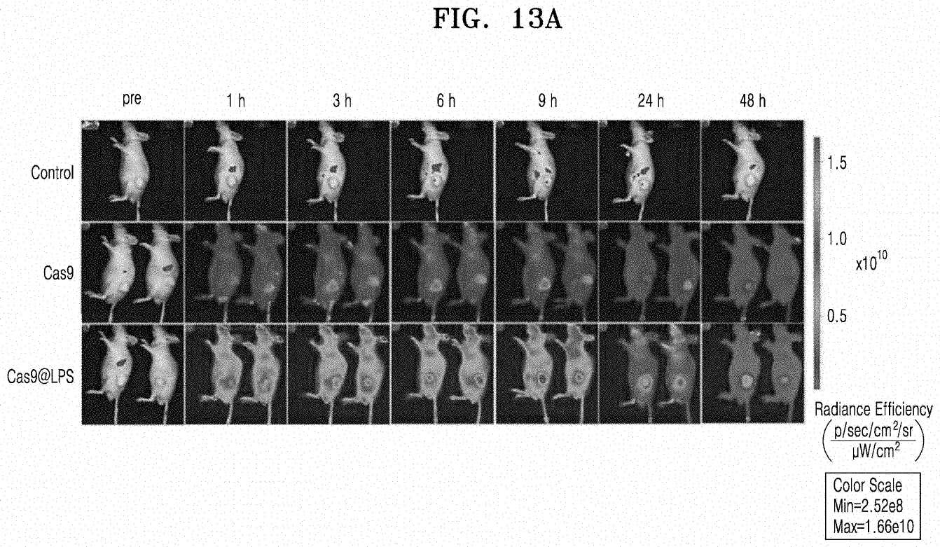

FIG. 13A shows real-time whole-body images after intravenous injection with Cas9-RNP or Cas9-RNP@LPS. FIG. 13B shows TEM images.

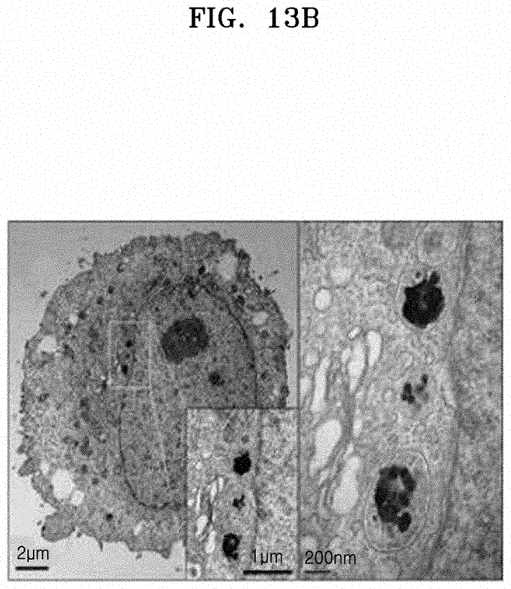

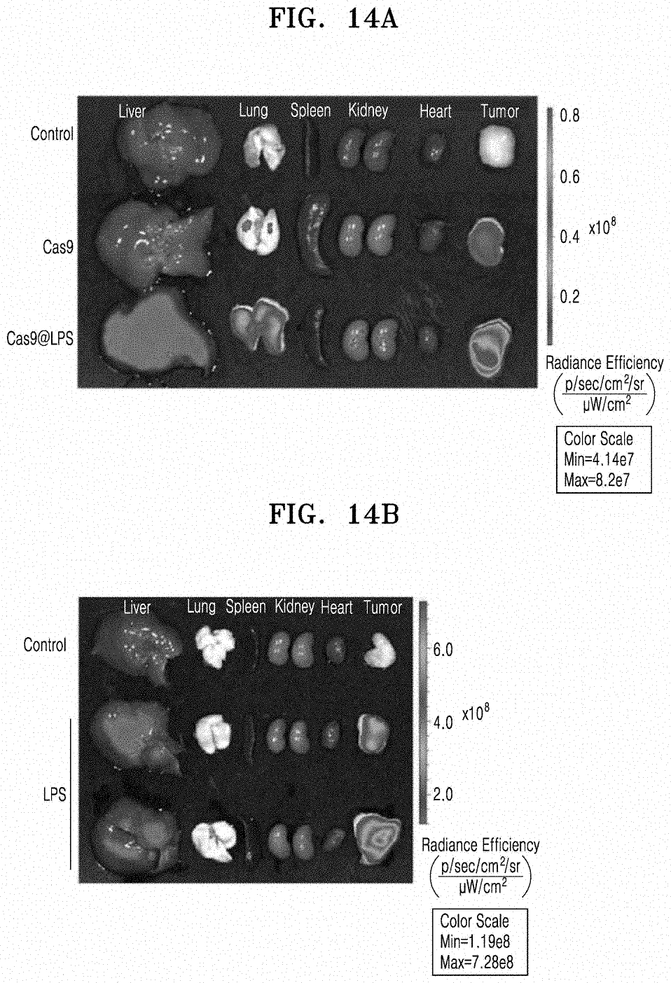

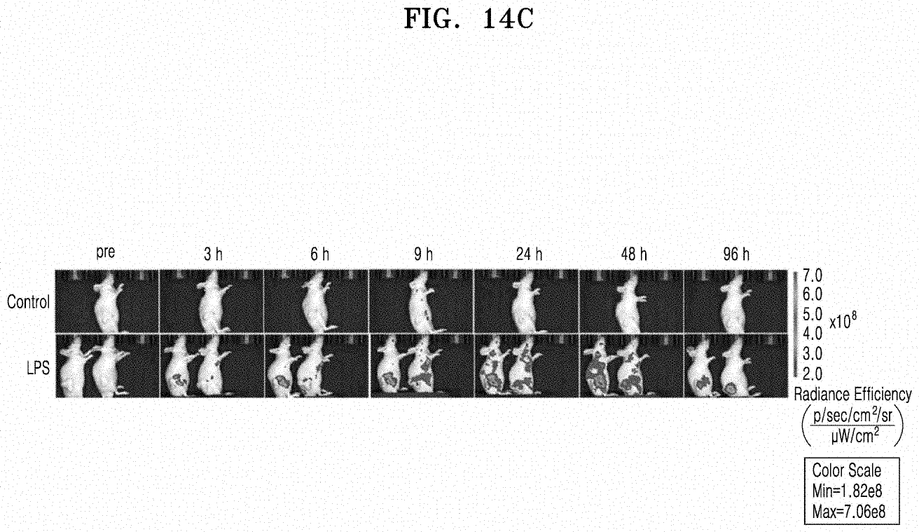

FIG. 14A shows fluorescence signals in liver, lung, spleen, kidney, heart, and tumor after intravenous injection with Cas9-RNP or Cas9-RNP@LPS. FIG. 14B shows fluorescence signals in liver, lung, spleen, kidney, heart, and tumor after intravenous injection with Cas9-RNP or Cas9-RNP@LPS. FIG. 14C shows fluorescence signals in mouse whole bodies after intravenous injection with Cas9-RNP or Cas9-RNP@LPS.

As shown in FIG. 13A and FIG. 14A to FIG. 14C, the fluorescence signals of Cas9 protein and LPS were strongly observed in tumors, but slightly detected in the liver and lung. Moreover, the fluorescence signal in the free Cas9-treated tumor disappeared due to the degradation and removal of the Cas9 protein. However, the fluorescence signal in the Cas9-RNP@LPS-treated tumor remained over 2 days. This result indicated that LPS enables the sustained release of Cas9-RNP and maintains Cas9 protein activity with high stability.

Further, as shown in FIG. 13B, the TEM image showed the intracellular localization of the Cas9-RNP@LPS complex in the perinuclear region.

Collectively, these results demonstrated the tumor-specific intracellular delivery of Cas9-RNP with long-lasting activity by Cas9-RNP@LPS. Therefore, the results showed that the undesired side-effect may be minimized and the gene editing efficacy may also be maximized.

(2) Evaluation of Anti-Tumor Activity of Cas9-RNP by Genome Editing in Multiple Genes

Next, the present inventors investigated the anti-tumor activity of Cas9-RNP by genome editing in multiple genes (FGFR3 and TACC3) in human cancer xenograft mice model. Each tumor was grown up to 100 mm³ in volume and then treated with Cas9-RNP/Lipo, Cas9-RNP@LPS (6 mg mL⁻¹) or PBS by intravenous injection. The changes in tumor volume in each group was monitored for 15 days.

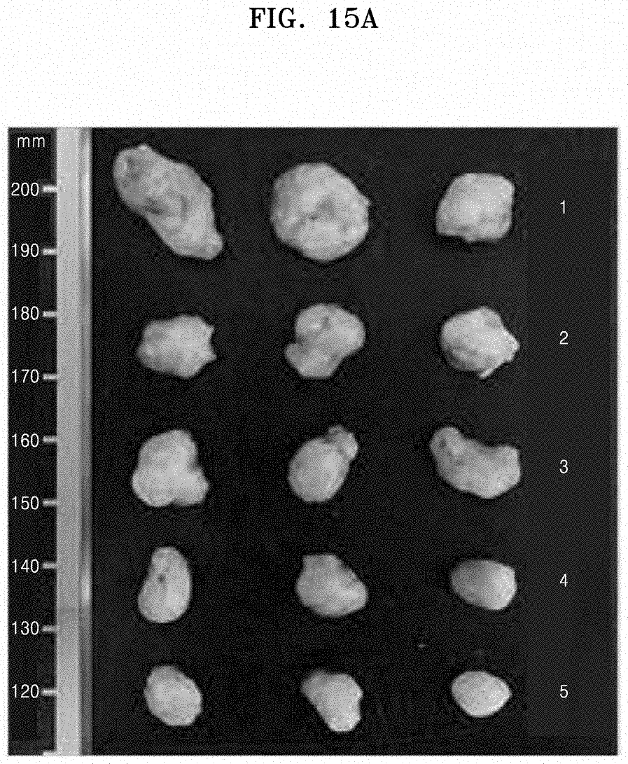

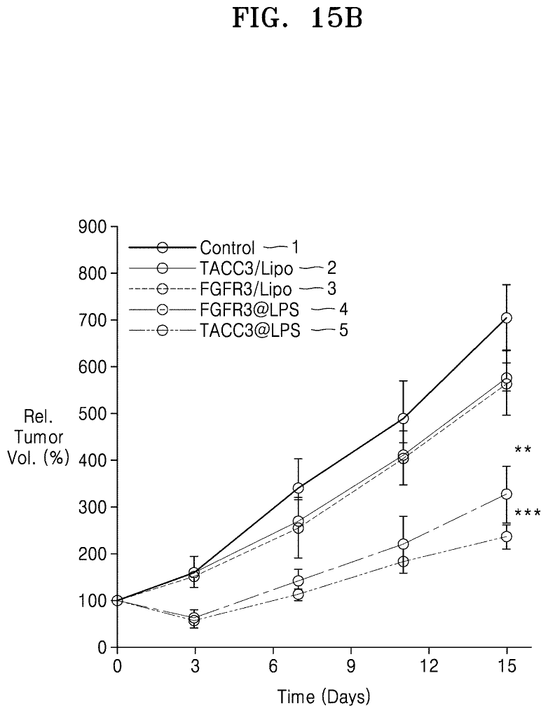

FIGS. 15A-15B show anti-tumor activity of Cas9-RNP by genome editing of multiple genes (FGFR3 and TACC3) in a human cancer xenograft mice model, **p<0.01, ***p<0.001.

As shown in FIGS. 15A-15B, tumor growth in the mice treated with Cas9-RNP@LPS targeting FGFR3 (FGFR3@LPS) and TACC3 (TACC3@LPS) were significantly suppressed. However, those in other groups treated with Cas9-RNP/Lipo (FGFR3/Lipo and TACC3/Lipo) showed negligible difference in tumor volume as compared to control groups.

Based on the statistically significant difference in tumors from various groups, hematoxylin and eosin staining of tumor sections was performed at 15 days after treatment.

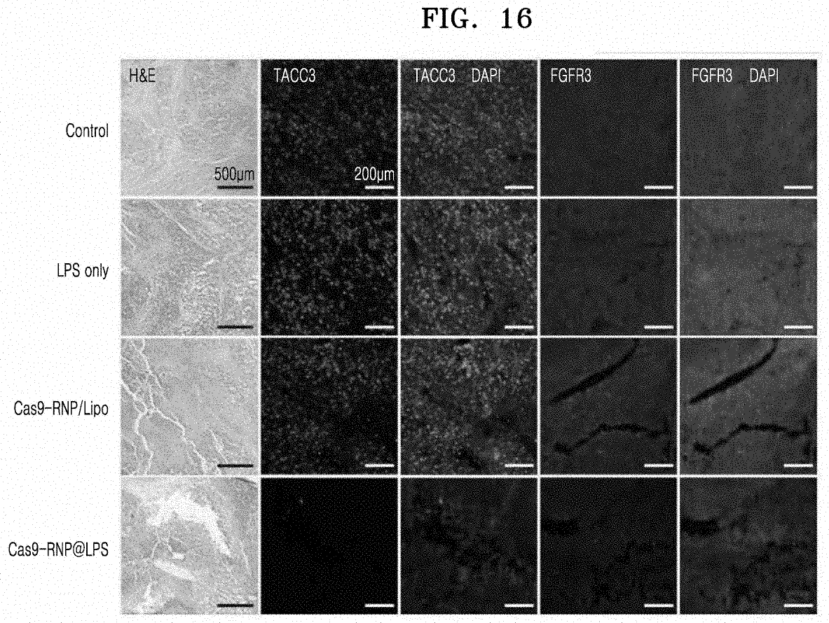

FIG. 16 shows hematoxylin and eosin staining results showing intensive apoptosis only in tumors treated with Cas9-RNP@LPS.

FIG. 17 shows histological images showing viability of major organs after injection with Cas9-RNP@LPS.

As shown in FIG. 16, the histological image showed extensive apoptosis only in tumors treated with Cas9-RNP@LPS, which was significantly in contrast to that observed with Cas9-RNP/Lipo, LPS alone, and 1×PBS-treated groups.

Moreover, as shown in FIG. 17, no remarkable systemic toxicity was observed in the histological sections from major organs of mice treated with the Cas9-RNP@LPS complex.

These results indicate that LPS enables safe delivery of Cas9-RNP and promotes site-specific gene modification, while mitigating any potential undesirable side-effect in normal tissue.

To precisely investigate the knockout of multiple genes by Cas9-RNP@LPS, the sectioned tissue excised from each mouse was evaluated using immunohistochemistry.

As shown in FIG. 16, fluorescence images showed a notable decrease in fluorescence of both FGFR3 (green) and TACC3 (red) in sectioned tissues treated with Cas9-RNP@LPS complex as compared to those treated with Cas9-RNP/Lipo, LPS alone, and 1×PBS only. This result suggests the successful knockout of target gene expression via LPS-mediated gene editing in the mouse xenograft model.

Collectively, these results demonstrated that Cas9-RNP@LPS effectively induced cell death and inhibited the growth of tumors by target-specific genome editing in vivo without systemic toxicity.

### CONCLUSION

The present inventors have developed a robust LPS-based Cas9-RNP delivery platform for targeting multiple genes that are highly relevant for the fusion gene of cancer. LPS exhibited a high loading capacity of Cas9-RNP and durable enzymatic activity, as well as efficient cellular permeability, endosomal escape, and release of Cas9-RNP from LPS to the cytoplasm.

Compared to lipid-based classical methods for direct cytosolic delivery of Cas9-RNP, the LPS-mediated Cas9-RNP delivery system according to one embodiment showed over 50% gene editing ability for multiple target genes in vitro, as evident from the results of NGS analysis and gene expression at transcription and translation levels. Furthermore, the LPS delivery system showed robust gene editing outcome and tumor-specific accumulation as well as retained enzyme activity and stability, which represents efficacy and safety in vivo.

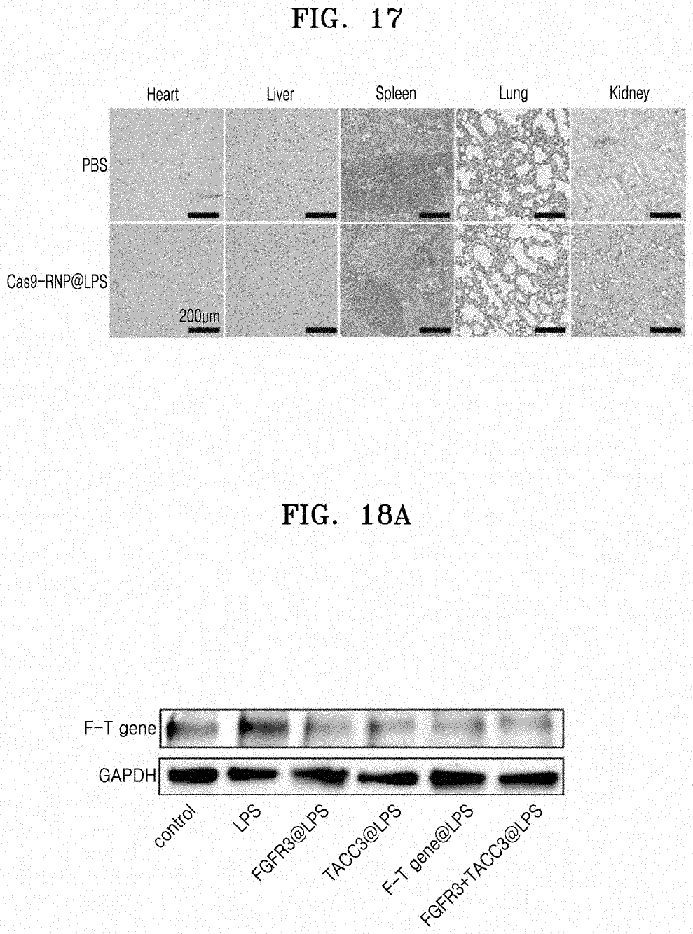

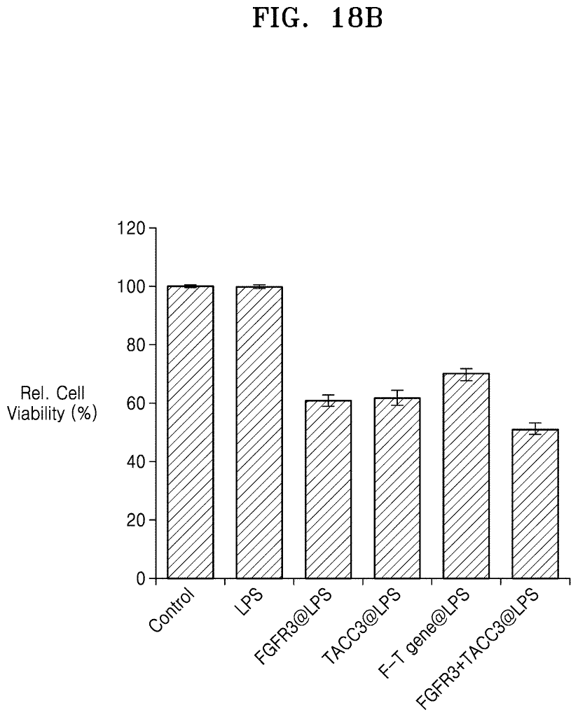

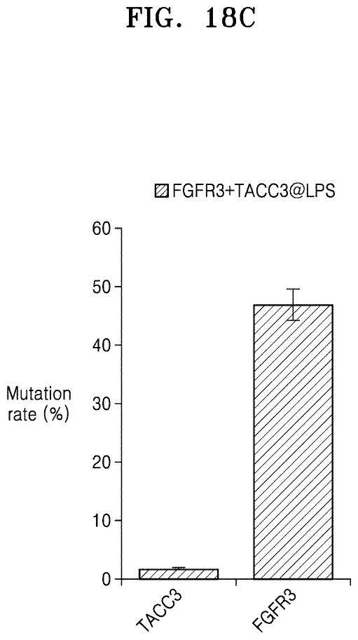

FIG. 18A to FIG. 18C show results of a comparison of targeting F-T fusion gene and multiple genes having high similarity to the F-T fusion gene.

As shown in FIG. 18A to FIG. 18C, even when half the amount of sgRNA targeting multiple genes was delivered simultaneously by LPS, the fusion gene was knockout out with high efficiency, and the corresponding cell death effect and mutation efficiency were shown. In the end, LPS enabled multigene engineering with high specificity. Thus, this suggests new alternative approaches to co-editing multiple genes to obtain a potential inhibitor of fusion genes.

Owing to its porous structure with cavity and surface functionality, LPS may provide modularity and controllable delivery of multi-cargo, which is a major benefit over existing methods, which show less pliability.

The present inventors anticipate that the LPS-based Cas9-RNP delivery platform will be promising as a unique and modular delivery system for CRISPR/Cas9-mediated in vivo genome editing, as well as in other editing systems such as homology-directed repair for different therapeutic purposes. In addition, it will be obvious that the LPS-based platform can be used as a delivery system for various gene-editing materials such as Cas9-protein as well as Base editor-RNP.

According to a preparation method of an aspect, it is possible to prepare a surface-aminated mesoporous silica nanoparticle having a large pore size.

A surface-aminated mesoporous silica nanoparticle of another aspect may be loaded with a large ribonucleoprotein, such as Cas protein-RNP.

Therefore, the surface-aminated mesoporous silica nanoparticle may be used as a Cas protein-RNP delivery system to be applied to treatment of various diseases treatable by gene editing.

It should be understood that embodiments described herein should be considered in a descriptive sense only and not for purposes of limitation. Descriptions of features or aspects within each embodiment should typically be considered as available for other similar features or aspects in other embodiments. While one or more embodiments have been described with reference to the figures, it will be understood by those of ordinary skill in the art that various changes in form and details may be made therein without departing from the spirit and scope of the disclosure as defined by the following claims.

What is claimed is:

1. A surface-aminated mesoporous silica nanoparticle which has a mean pore size of about 15 nm to about 50 nm and is capable of being loaded with ribonucleoprotein (RNP).

2. The surface-aminated mesoporous silica nanoparticle of claim 1, wherein the nanoparticle has a diameter of about 150 nm to about 1000 nm.

3. The surface-aminated mesoporous silica nanoparticle of claim 1, wherein the nanoparticle has a zeta potential of about +20.0 mV to about +30.0 mV.

4. The surface-aminated mesoporous silica nanoparticle of claim 1, wherein the nanoparticle has an RNP loading capacity of about 10% (w/w) to about 20% (w/w) at pH 7.4.

5. The surface-aminated mesoporous silica nanoparticle of claim 1, wherein the nanoparticle has a higher drug release rate under an acidic pH condition of pH 6.5 or less than a drug release rate of the nanoparticle under a neutral pH condition of pH 7.0 to pH 8.0.

6. The surface-aminated mesoporous silica nanoparticle of claim 1, wherein the nanoparticle exhibits a drug release rate of 5% or less under a neutral pH condition of pH 7.0 to pH 8.0 for 12 hours, and a drug release rate of 30% or more under an acidic pH condition of pH 6.5 or less for 12 hours.

7. The surface-aminated mesoporous silica nanoparticle of claim 1, wherein the nanoparticle releases RNP to cytoplasm in vivo.

8. The surface-aminated mesoporous silica nanoparticle of claim 1, wherein the RNP is a Cas protein-RNP.

9. The surface-aminated mesoporous silica nanoparticle of claim 8, wherein the Cas protein of the Cas protein-RNP is Cas9, nickase Cas9 (nCas9), deactivated Cas9 (dCas9), nCas9 fused to cytidine deaminase, nCas9 fused to adenine deaminase, dCas9 fused to cytidine deaminase, dCas9 fused to adenine deaminase, Cas12, Cas13, Cpf1, C2c2, or a combination of two or more thereof.

10. The surface-aminated mesoporous silica nanoparticle of claim 8, wherein the Cas protein-RNP is Cas9-RNP or Base editor-RNP.

11. The surface-aminated mesoporous silica nanoparticle of claim 10, wherein the Cas protein-RNP is one type or two or more types.

12. The surface-aminated mesoporous silica nanoparticle of claim 10, wherein the Cas9-RNP is two types, wherein any one type thereof comprises sgRNA targeting fibroblast growth factor receptor 3 (FGFR3), and
the other type thereof comprises sgRNA targeting transforming acidic coiled-coil containing protein 3 (TACC3).

13. The surface-aminated mesoporous silica nanoparticle of claim 12, wherein the sgRNA targeting FGFR3 comprises any one nucleotide sequence of SEQ ID NOS: 3 to 5 and
the sgRNA targeting TACC3 comprises any one nucleotide sequence of SEQ ID NOS: 6 to 8.

14. The surface-aminated mesoporous silica nanoparticle of claim 1, wherein the surface-aminated mesoporous silica nanoparticle is prepared by a method comprising:
(1) dissolving a cationic surfactant in a basic solution;
(2) adding a silica precursor solution to the solution prepared in (1) and stirring the mixture to prepare a porous silica nanoparticle;
(3) adding a benzenoid compound solution to the porous silica nanoparticle and allowing the same to react to prepare a mesoporous silica nanoparticle; and
(4) adding an amine compound solution to the mesoporous silica nanoparticle to prepare a mixture, and refluxing the mixture to prepare the surface-aminated mesoporous silica nanoparticle.

15. The surface-aminated mesoporous silica nanoparticle of claim 14,
wherein in (1), the cationic surfactant is cetyl trimethyl ammonium bromide (CTAB) or cetyl trimethyl ammonium chloride (CTAC);
in (2), the silica precursor is tetramethyl orthosilicate (TMOS) or tetraethyl orthosilicate (TEOS);
in (3), the benzenoid compound is any one or more selected from the group consisting of benzene, trimethylbenzene (TMB), 1,3,5-triethylbenzene (TEB), 1,3,5-tri-tert-butylbenzene (TtBB), and 1,3,5-Triisopropylbenzene (TiPB); and
in (4), the amine compound is any one or more selected from the group consisting of 3-aminopropyltriethoxysilane (APTES), N(beta-aminoethyl)gamma-aminopropylmethyldimethoxysilane (AEAPMDMS), N-[3-(trimethoxysilyl)propyl]ethylenediamine, N1-(3-trimethoxysilylpropyl)diethylenetriamine, (3-aminopropyl)trimethoxysilane, N-[3-(trimethoxysilyl)propyl]aniline, trimethoxy[3-(methylamino)propyl]silane, 3-(2-aminoethylamino)propyldimethoxymethylsilane, trimethoxysilylpropyl modified polyethylenimine (TPPI), p-aminophenyltrimethoxysilane, and 1-amino-2-(dimethylethoxysilyl)propane.

16. A pharmaceutical composition for preventing or treating cancer, the pharmaceutical composition comprising the surface-aminated mesoporous silica nanoparticle of claim 1,
wherein Cas9-RNP loaded onto the nanoparticle is two types, and any one thereof comprises sgRNA targeting fibroblast growth factor receptor 3 (FGFR3), and the other type thereof comprises sgRNA targeting transforming acidic coiled-coil containing protein 3 (TACC3).

17. The pharmaceutical composition of claim 16, wherein
the sgRNA targeting FGFR3 comprises any one nucleotide sequence of SEQ ID NOS: 3 to 5, and
the sgRNA targeting TACC3 comprises any one nucleotide sequence of SEQ ID NOS: 6 to 8.

18. The pharmaceutical composition of claim 16, wherein the cancer is selected from the group consisting of glioblastoma, lung adenocarcinoma, squamous cell lung carcinoma, head and neck squamous cell carcinoma, bladder cancer, cervical cancer, breast invasive ductal carcinoma, urothelial carcinoma, malignant glioma, and glioma.

19. A method of preventing or treating a disease, the method comprising administering an effective amount of the pharmaceutical composition of claim 16 to a subject in need thereof.
